(19) United States
(12) Patent Application Publication
(10) Pub. No.: US 2024/0081351 A1
(43) Pub. Date: Mar. 14, 2024

(54) ENZYMATIC MODIFICATION OF PHOSPHOLIPIDS IN FOOD

(71) Applicant: DUPONT NUTRITION BIOSCIENCES APS, Kongens Lyngby (DK)

(72) Inventors: Keya Zhang, Shanghai (CN); Lone Broend Miller, Viby (DK); Lene Kragh, Copenhagen (DK); Tina Lillan Joergensen, Silkeborg (DK)

(21) Appl. No.: 18/351,069

(22) Filed: Jul. 12, 2023

Related U.S. Application Data
(63) Continuation of application No. 16/954,367, filed on Jun. 16, 2020, now Pat. No. 11,744,254.

(30) Foreign Application Priority Data
Dec. 19, 2017 (WO) PCT/CN2017/117174

(51) Int. Cl. A21D 8/04

(52) U.S. Cl. CPC A21D 8/042; C12Y 301/01032

(57) ABSTRACT

A phospholipase A1 characterized by having an sn1/sn2 specificity ratio of about 55/45 or greater wherein said phospholipase A1 has a lysophospholipase/phospholipase activity ratio of less than 0.02 is presented in conjunction with methods for use in lipid containing food matrix, baking and making dough with the phospholipase and also including baking improvers using the disclosed phospholipase A1.

### CROSS-REFERENCE TO RELATED APPLICATIONS

This application is a continuation of U.S. patent application Ser. No. 16/954,367, filed Jun. 16, 2020, which claims priority under 35 USC § 371 as a national stage of International Patent Application No. PCT/EP2018/085339, filed Dec. 17, 2018, which claims priority to International Patent Application No. PCT/CN2017/117174, filed Dec. 19, 2017, the contents of which are incorporated herein by reference in their entireties.

### INCORPORTATION BY REFERENCE OF THE SEQUENCE LISTING

The sequence listing provided in the file named NB41421USPCN2_SequenceListing.xml with a size of 31 KB which was created on Jul. 7, 2023, and which is filed herewith, is incorporated by reference herein in its entirety.

### TECHNICAL FIELD

The present invention relates to phospholipases and their use in the manufacture of food. The present invention further relates to methods of making dough and baked products using phospholipases.

### BACKGROUND

The use of lipases in bread dough is well known. For example, in EP0585988 it is shown that the addition of lipase to dough provided an anti-staling effect in bread baked therefrom. WO94/04035 teaches that an improved softness can be obtained by adding a lipase to dough. It has also been shown that exogenous lipases can modify bread volume.

While lipases, including phospholipases, have been described for their positive properties in the preparation of dough and baked products, the performance of prior art lipases has many drawbacks because prior art lipases have generally had multiple activities, reducing or eliminating the potential beneficial effect of the lipase. Therefore, today, there is still a need in some food applications, in particular, in baking, for improved lipases having higher specificity.

### SUMMARY OF THE INVENTION

In accordance with an aspect of the present invention, an isolated polypeptide comprising a phospholipase A1 characterized by having an sn1/sn2 specificity ratio of about 55/45 or greater wherein said phospholipase A1 has a lysophospholipase/phospholipase activity ratio of less than 0.01 is presented. Optionally, the sn1/sn2 specificity ratio is about 60/40, 70/30, 80/20, 90/10, 95/5 or 99/1. Optionally, the sn1/sn2 specificity ratio is about 74/26.

Optionally, the lysophospholipase/phospholipase activity ratio is less than 0.009, 0.008, 0.007, 0.006, 0.005, 0.004, 0.003, 0.002 or 0.001. Optionally, the lysophospholipase/phospholipase activity ratio is less than 0.001 and the sn1/sn2 specificity ratio is about 60/40, 70/30, 80/20, 90/10, 95/5 or 99/1. Optionally, the lysophospholipase/phospholipase activity ratio is less than 0.001 and the sn1/sn2 specificity ratio is about 74/26.

Optionally, the phospholipase A1 is an enzyme comprising a protein sequence having at least 80% sequence identity SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. Optionally, the phospholipase A1 is an enzyme comprising a protein sequence having at least 80% sequence identity to SEQ ID NO: 6

Optionally, the phospholipase A1 is an enzyme comprising a protein sequence having at least 90% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. Optionally, the phospholipase A1 is an enzyme comprising a protein sequence having at least 90% sequence identity to SEQ ID NO: 6.

Optionally, the phospholipase A1 is an enzyme comprising a protein sequence having at least 95% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. Optionally, the phospholipase A1 is an enzyme comprising a protein sequence having at least 95% sequence identity to SEQ ID NO: 6.

Optionally, the phospholipase A1 is an enzyme comprising a protein sequence having 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. Optionally, the phospholipase A1 is an enzyme comprising a protein sequence having 100% sequence identity to SEQ ID NO: 6.

In another aspect of the present invention, a method is presented of making a dough, the method comprising admixing a dough component selected from the group consisting of flour, salt, water, sugar, fat, lecithin, oil and yeast with an isolated polypeptide comprising a phospholipase A1 characterized by having an sn1/sn2 specificity ratio of about 55/45 or greater wherein said phospholipase A1 has a lysophospholipase/phospholipase activity ratio of less than 0.01. Optionally, the sn1/sn2 specificity ratio is about 60/40, 70/30, 80/20, 90/10, 95/5 or 99/1. Optionally, the sn1/sn2 specificity ratio is about 74/26.

Optionally, the lysophospholipase/phospholipase activity ratio is less than 0.009, 0.008, 0.007, 0.006, 0.005, 0.004, 0.003, 0.002 or 0.001. Optionally, the lysophospholipase/phospholipase activity ratio is less than 0.001 and the sn1/sn2 specificity ratio is about 60/40, 70/30, 80/20, 90/10, 95/5 or 99/1. Optionally, the lysophospholipase/phospholipase activity ratio is less than 0.001 and the sn1/sn2 specificity ratio is about 74/26.

In another aspect of the present invention, a dough is presented comprising a phospholipase A1 enzyme characterized by having an sn1/sn2 specificity ratio of about 55/45 or greater wherein the phospholipase A1 has a lysophospholipase/phospholipase activity ratio of less than 0.01. Optionally, the dough has improved extensibility and/or stability.

In another aspect of the present invention, a method of preparing a baked product is presented in which a dough as described above is baked. In another aspect of the present invention, a baked product is presented. Optionally, the baked product has at least one improved property selected from the group consisting of improved crumb pore size, improved uniformity of gas bubbles, no separation between crust and crumb, increased volume, increased crust crispiness and improved oven spring. Optionally, the improved property is increased crust crispiness.

In another aspect of the present invention, a pre-mix for baking is presented comprising flour and a phospholipase A1 enzyme characterized by having an sn1/sn2 specificity ratio of about 55/45 or greater wherein the phospholipase A1 has a lysophospholipase/phospholipase activity ratio of less than 0.01. In another aspect of the present invention, a baking improver is presented comprising a granulate or agglomerated powder comprising a phospholipase A1 enzyme characterized by having an sn1/sn2 specificity ratio of about 55/45 or greater wherein the phospholipase A1 has a lysophospholipase/phospholipase activity ratio of less than 0.01.

In another aspect of the present invention, a method of making a dough is presented as set forth above but in which at least one additional enzyme useful for improving dough and/or a baked product made therefrom is included. Optionally, the additional enzyme is selected from the group consisting of amylase, cyclodextrin glucanotransferase, peptidase, transglutaminase, lipase, galactolipase, phospholipase which is different from said phospholipase A1, cellulase, hemicellulase, protease, protein disulfide isomerase, glycosyltransferase, peroxidase, lipoxygenase, laccase, and oxidase. Optionally, the amylase is an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the additional enzyme has galactolipase activity. Optionally, the additional enzyme is a phospholipase comprising SEQ ID NO: 17and/or SEQ ID NO: 18.

In another aspect of the present invention, a method for modification of a phospholipid emulsifier comprising treatment of the emulsifier with a phospholipase A1 enzyme characterized by having an sn1/sn2 specificity ratio of about 55/45 or greater wherein the phospholipase A1 has a lysophospholipase/phospholipase activity ratio of less than 0.01. Optionally, the phospholipid emulsifier is lecithin.

In another aspect of the present invention, a method of creating a lysophospholipid in a lipid containing food matrix is presented comprising adding to the lipid containing food matrix a phospholipase A1 enzyme characterized by having an sn1/sn2 specificity ratio of about 55/45 or greater wherein the phospholipase A1 has a lysophospholipase/phospholipase activity ratio of less than 0.01. Optionally, the lipid containing food matrix is selected from the group consisting of eggs and food products containing eggs such as dough for sweet bakery goods, processed meat, milk based products, vegetable oil and sweet bakery goods, including cakes and cookies.

### BRIEF DESCRIPTION OF THE BIOLOGICAL SEQUENCES

SEQ ID NO: 1 sets forth the full length amino acid sequence of the phospholipase variant from Trichoderma harzianum (full length CRC08310).
SEQ ID NO: 2 sets forth the predicted, mature amino acid sequence of the phospholipase variant from Trichoderma harzianum (predicted mature CRC08310).
SEQ ID NO: 3—sets forth the full length amino acid sequence of the phospholipase variant from Pestalotiopsis fici (full length CRC08316).
SEQ ID NO: 4 sets forth the predicted, mature amino acid sequence of the phospholipase variant from Pestalotiopsis fici (predicted mature CRC08316).
SEQ ID NO: 5—sets forth the full length amino acid sequence of the phospholipase variant from Metarhizium guizhouense (full length CRC08319).
SEQ ID NO: 6 sets forth the predicted, mature amino acid sequence of the phospholipase variant from Metarhizium guizhouense (predicted mature CRC08319).
SEQ ID NO: 7—sets forth the full length amino acid sequence of the phospholipase variant from Diaporthe ampelina (full length CRC08405).
SEQ ID NO: 8: sets forth the predicted, mature amino acid sequence of the phospholipase variant from Diaporthe ampelina (predicted mature CRC08405).
SEQ ID NO: 9—sets forth the full length amino acid sequence of the phospholipase variant from Magnaporthe oryzae (full length CRC08418).
SEQ ID NO: 10 sets forth the predicted, mature amino acid sequence of the phospholipase variant from Magnaporthe oryzae (predicted mature CRC08418).
SEQ ID NO: 11—sets forth the full length amino acid sequence of the phospholipase variant from Neonectria ditissima (full length CRC08826).
SEQ ID NO: 12 sets forth the predicted, mature amino acid sequence of the phospholipase variant from Neonectria ditissima (predicted mature CRC08826).
SEQ ID NO: 13—sets forth the full length amino acid sequence of the phospholipase variant from Trichoderma gamsii (full length CRC08833).
SEQ ID NO: 14 sets forth the predicted, mature amino acid sequence of the phospholipase variant from Trichoderma gamsii (predicted mature CRC08833).
SEQ ID NO: 15:—sets forth the full length amino acid sequence of the phospholipase variant from Metarhizium anisopliae (full length CRC08845).
SEQ ID NO: 16 sets forth the predicted, mature amino acid sequence of the phospholipase variant from Metarhizium anisopliae (predicted mature CRC08845).
SEQ ID NO: 17—sets forth the mature amino acid sequence of a phospholipase A1 used in the commercial product Powerbake 4080.
SEQ ID NO: 18—sets forth the full length amino acid sequence of a phospholipase A1 used in the commercial product Lipopan F.
SEQ ID NO: 19—sets forth the codon-optimized synthetic nucleic acid sequence of full-length CRC08310.
SEQ ID NO: 20—sets forth codon-optimized synthetic nucleic acid sequence of full-length CRC08316.
SEQ ID NO: 21—sets forth codon-optimized synthetic nucleic acid sequence of full-length CRC08319.
SEQ ID NO: 22—sets forth codon-optimized synthetic nucleic acid sequence of full-length CRC08405.
SEQ ID NO: 23—sets forth codon-optimized synthetic nucleic acid sequence of full-length CRC08418.
SEQ ID NO: 24—sets forth codon-optimized synthetic nucleic acid sequence of full-length CRC08826.
SEQ ID NO: 25—sets forth codon-optimized synthetic nucleic acid sequence of full-length CRC08833.
SEQ ID NO: 26—sets forth codon-optimized synthetic nucleic acid sequence of full-length CRC08845.

### DESCRIPTION OF FIGURES

FIG. 1A depicts crusty roll specific volume (ccm/g) presented as a function of optimal dosage of Lipopan F (relative dosing based on mg protein/kg flour).

FIG. 1B depicts crusty roll specific volume (ccm/g) presented as a function of dosage of CRC08319.

FIG. 2A depicts dough lipid profiling using Lipopan F.

FIG. 2B depicts dough lipid profiling using CRC08319.

### DETAILED DESCRIPTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Abbreviations

NAPE—N-acyl phosphatidylethanolamine
NALPE—N-acyl lysophosphatidylethanolamine
NAGPE—N-acyl glycerophosphoethanolamine
DGDG—digalactosyldiglyceride
DGMG—digalactosylmonoglyceride
MGDG—monogalactosyldiglyceride
MGMG—monogalactosylmonoglyceride
PC—phosphatidylcholine
LPC—lysophosphatidylcholine
PLA—phospholipase A

### Definitions

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a phospholipase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptide, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min, 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a phospholipase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein. Alternatively, specific activity can refer to the number of moles of product generated by an enzyme of enzyme preparation per unit of time under specific conditions.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty: OFF

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the term “lipase” refers to triacylglycerol lipases as defined by enzyme entry EC 3.1.1.3. Lipases catalyse the hydrolysis of triacylglycerols to give free fatty acids (saturated or unsaturated), diacylglyerols, monoacylglycerols and glycerol.

As used herein, the term “phospholipase” refers to an enzyme that hydrolyses phospholipids into fatty acids (saturated or unsaturated), lysophospholipids, diacylgycerols, choline phosphate and phophatidates, depending on the site of hydrolysis. Phospholipases are further classified into types A, B, C and D.

As used herein, the term “phospholipase A” refers to enzymes that catalyse the hydrolysis of the ester bond of the fatty acid components of phospholipids. There are two different types of phospholipase A activity that can be distinguished. Phospholipase A1, as defined in enzyme entry EC 3.1.1.32, and phospholipase A2, as defined in enzyme entry EC 3.1.1.4, catalyse the deacylation of one fatty acyl group in the sn1 and sn2 positions, respectively, from a diacylglycerophospholipid to produce lysophospholipid.

Phospholipase A1 and A2 catalyze the deacylation of one fatty acid group in the sn1 and sn2 positions, respectively. Hence, phospholipase A1 (also sometimes referred to herein as PLA1) hydrolyzes the 1-acyl group of a phospholipid, hydrolyzing the bond between the fatty acid and the glycerin residue at the one position. Phospholipase A2 (also sometimes referred to herein as PLA2) catalyzes hydrolysis of the 2-acyl group.

Hydrolysis of a phospholipid by a phospholipase produces a compound termed a lysophospholipid. Thus, selective hydrolysis of a phospholipid with a phospholipase A1 produces a 2-acyl lysophospholipid. Hydrolysis of a phospholipid with a phospholipase A2 produces a 1-acyl lysophospholipid. Another phospholipase is a “lysophospholipase” which catalyzes the hydrolysis of the remaining fatty acyl group in the lysophospholipid.

A used herein, the phrase “an sn1/sn2 specificity ratio” is defined here as the relative PLA1 activity divided by the relative PLA2 activity as set forth more fully below.

As used herein, the phrase “a lysophospholipase/phospholipase activity ratio” means (LPC-U/mg protein)/(PC-U/mg protein) as set forth more fully below.

Other definitions are set forth below.

### Additional Mutations

In some embodiments, the present phospholipases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present phospholipases may include any number of conservative amino acid substitutions. Exemplary conservative amino acid substitutions are listed in Table 1.

TABLE 1
Conservative amino acid substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo- |
| | | Arg, Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, |
| | | Trp, D-Trp, Trans-3,4, or 5-phenylproline, |
| | | cis-3, 4, or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present phospholipase may be “precursor,” “immature,” or “full-length,” in which case they include a signal sequence, or “mature,” in which case they lack a signal sequence and may be further truncated at the N- and/or C-terminus by proteolytic and/or non-proteolytic processing. In general, the mature forms of the polypeptides are generally the most useful. Unless otherwise noted, the amino acid residue numbering used herein refers to the mature forms of the respective phospholipase polypeptides. The present phospholipase polypeptides may also be truncated to remove the N or C-termini, so long as the resulting polypeptides retain phospholipase activity. In addition, phospholipase enzymes may be active fragments derived from a longer amino acid sequence. Active fragments are characterized by retaining some or all of the activity of the full length enzyme but have deletions from the N-terminus, from the C-terminus or internally or combinations thereof.

The present phospholipase may be a “chimeric” or “hybrid” polypeptide, in that it includes at least a portion of a first phospholipase polypeptide, and at least a portion of a second phospholipase polypeptide. The present phospholipase may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

### Production of Variant Phospholipases

The present phospholipase can be produced in host cells, for example, by secretion or intracellular expression. A cultured cell material (e.g., a whole-cell broth) comprising a phospholipase can be obtained following secretion of the phospholipase into the cell medium. Optionally, the phospholipase can be isolated from the host cells, or even isolated from the cell broth, depending on the desired purity of the final phospholipase. A gene encoding a phospholipase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, E. Coli.

The host cell further may express a nucleic acid encoding a homologous or heterologous phospholipase, i.e., a phospholipase that is not the same species as the host cell, or one or more other enzymes. The phospholipase may be a variant phospholipase. Additionally, the host may express one or more accessory enzymes, proteins, peptides.

### Vectors

A DNA construct comprising a nucleic acid encoding a phospholipase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding phospholipase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a phospholipase can be transformed and replicated in a bacterial host cell as a means of propagating and amplifying the vector. The vector also may be transformed into an expression host, so that the encoding nucleic acids can be expressed as a functional phospholipase. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). A representative vector is pJG153, a promoterless Cre expression vector that can be replicated in a bacterial host. See Harrison et al. (June 2011) Applied Environ. Microbiol. 77: 3916-22. pJG153 can be modified with routine skill to comprise and express a nucleic acid encoding a phospholipase.

A nucleic acid encoding a phospholipase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a phospholipase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a phospholipase is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from Trichoderma reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the phospholipase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant phospholipase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of phospholipase for subsequent enrichment or purification. Extracellular secretion of phospholipase into the culture medium can also be used to make a cultured cell material comprising the isolated phospholipase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the phospholipase to a host cell organelle such as a peroxisome, or to a particular host cell compartment. Such a targeting sequence includes but is not limited to the sequence, SKL. For expression under the direction of control sequences, the nucleic acid sequence of the phospholipase is operably linked to the control sequences in proper manner with respect to expression.

The procedures used to ligate the DNA construct encoding a phospholipase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

### Transformation and Culture of Host Cells

An isolated cell, either comprising a DNA construct or an expression vector, is advantageously used as a host cell in the recombinant production of a phospholipase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A phospholipase expressed by a fungal host cell can be glycosylated, i.e., will comprise a glycosyl moiety. The glycosylation pattern can be the same or different as present in the wild-type phospholipase. The type and/or degree of glycosylation may impart changes in enzymatic and/or biochemical properties.

It may be advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a phospholipase is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

### Expression

A method of producing a phospholipase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a phospholipase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a phospholipase. Fermentation may, therefore, be understood as comprising shake flask cultivation, small- or large-scale fermentation (including continuous, batch, fed-batch, or solid-state fermentations) in laboratory or industrial fermenters performed in a suitable medium and under conditions allowing the phospholipase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like. The polynucleotide encoding a phospholipase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a phospholipase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired phospholipase. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of a phospholipase.

### Methods for Enriching and Purifying Phospholipases

Fermentation, separation, and concentration techniques are well known in the art and conventional methods can be used in order to prepare a phospholipase polypeptide-containing solution.

After fermentation, a fermentation broth is obtained, the microbial cells and various suspended solids, including residual raw fermentation materials, are removed by conventional separation techniques in order to obtain a phospholipase solution. Filtration, centrifugation, microfiltration, rotary vacuum drum filtration, ultrafiltration, centrifugation followed by ultra-filtration, extraction, or chromatography, or the like, are generally used.

It is desirable to concentrate a phospholipase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

The enzyme solution is concentrated into a concentrated enzyme solution until the enzyme activity of the concentrated phospholipase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides. Exemplary metal halides include sodium chloride, potassium chloride, sodium bromide, potassium bromide and blends of two or more of these metal halides. The metal halide precipitation agent, sodium chloride, can also be used as a preservative.

The metal halide precipitation agent is used in an amount effective to precipitate a phospholipase. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific phospholipase polypeptide and on its concentration in the concentrated enzyme solution.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both preservative agents. For further descriptions, see, e.g., U.S. Pat. No. 5,281,526.

Addition of the organic compound precipitation agent provides the advantage of high flexibility of the precipitation conditions with respect to pH, temperature, phospholipase concentration, precipitation agent concentration, and time of incubation.

The organic compound precipitation agent is used in an amount effective to improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be enriched or purified. Generally, the pH is adjusted at a level near the isoelectric point of the phospholipase. The pH can be adjusted at a pH in a range from about 2.5 pH units below the isoelectric point (pI) up to about 2.5 pH units above the isoelectric point.

The incubation time necessary to obtain an enriched or purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of enriched or purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

During fermentation, a phospholipase polypeptide accumulates in the culture broth. For the isolation, enrichment, or purification of the desired phospholipase, the culture broth is centrifuged or filtered to eliminate cells, and the resulting cell-free liquid is used for enzyme enrichment or purification. In one embodiment, the cell-free broth is subjected to salting out using ammonium sulfate at about 70% saturation; the 70% saturation-precipitation fraction is then dissolved in a buffer and applied to a column such as a Sephadex G-100 column, and eluted to recover the enzyme-active fraction. For further enrichment or purification, a conventional procedure such as ion exchange chromatography may be used.

Enriched or purified enzymes can be made into a final product that is either liquid (solution, slurry) or solid (granular, powder).

### Description of the Preferred Embodiments

In accordance with an aspect of the present invention, an isolated polypeptide comprising a phospholipase A1 characterized by having an sn1/sn2 specificity ratio of about 55/45 or greater wherein said phospholipase A1 has a lysophospholipase/phospholipase activity ratio of less than 0.01 is presented. Preferably, the sn1/sn2 specificity ratio is about 60/40, 70/30, 80/20, 90/10, 95/5 or 99/1. In other preferred embodiments, the sn1/sn2 specificity ratio is about 74/26.

Preferably, the lysophospholipase/phospholipase activity ratio is less than 0.009, 0.008, 0.007, 0.006, 0.005, 0.004, 0.003, 0.002 or 0.001. In still more preferred embodiments, the lysophospholipase/phospholipase activity ratio is less than 0.001 and the sn1/sn2 specificity ratio is about 60/40, 70/30, 80/20, 90/10, 95/5 or 99/1. In still other preferred embodiments, the lysophospholipase/phospholipase activity ratio is less than 0.001 and the sn1/sn2 specificity ratio is about 74/26.

In other preferred embodiments, the phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. More preferably, the phospholipase A1 is an enzyme comprising a protein sequence having at least 80% sequence identity to SEQ ID NO: 6.

In other preferred embodiments, the phospholipase A1 is an enzyme comprising a protein sequence having at least 90% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. More preferably, the phospholipase A1 is an enzyme comprising a protein sequence having at least 90% sequence identity to SEQ ID NO: 6.

In other preferred embodiments, the phospholipase A1 is an enzyme comprising a protein sequence having at least 95% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. More preferably, the phospholipase A1 is an enzyme comprising a protein sequence having at least 95% sequence identity to SEQ ID NO: 6.

In other preferred embodiments, the phospholipase A1 is an enzyme comprising a protein sequence having 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. More preferably, the phospholipase A1 is an enzyme comprising a protein sequence having 100% sequence identity to SEQ ID NO: 6.

In another aspect of the present invention, a method is presented of making a dough, the method comprising admixing a dough component selected from the group consisting of flour, salt, water, sugar, fat, lecithin, oil and yeast with an isolated polypeptide comprising a phospholipase A1 characterized by having an sn1/sn2 specificity ratio of about 55/45 or greater wherein said phospholipase A1 has a lysophospholipase/phospholipase activity ratio of less than 0.01.

Preferably, the sn1/sn2 specificity ratio is about 60/40, 70/30, 80/20, 90/10, 95/5 or 99/1. In other preferred embodiments, the sn1/sn2 specificity ratio is about 74/26.

Preferably, the lysophospholipase/phospholipase activity ratio is less than 0.009, 0.008, 0.007, 0.006, 0.005, 0.004, 0.003, 0.002 or 0.001. In still more preferred embodiments, the lysophospholipase/phospholipase activity ratio is less than 0.001 and the sn1/sn2 specificity ratio is about 60/40, 70/30, 80/20, 90/10, 95/5 or 99/1. In still other preferred embodiments, the lysophospholipase/phospholipase activity ratio is less than 0.001 and the sn1/sn2 specificity ratio is about 74/26.

In other preferred embodiments, the phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16. More preferably, the phospholipase A1 is an enzyme comprising a protein sequence having at least 80% sequence identity to SEQ ID NO: 6.

In other preferred embodiments, the phospholipase A1 is an enzyme comprising a protein sequence having at least 90% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16. More preferably, the phospholipase A1 is an enzyme comprising a protein sequence having at least 90% sequence identity to SEQ ID NO: 6.

In other preferred embodiments, the phospholipase A1 is an enzyme comprising a protein sequence having at least 95% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16. More preferably, the phospholipase A1 is an enzyme comprising a protein sequence having at least 95% sequence identity to SEQ ID NO: 6.

In other preferred embodiments, the phospholipase A1 is an enzyme comprising a protein sequence having 100% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16. More preferably, the phospholipase A1 is an enzyme comprising a protein sequence having 100% sequence identity to SEQ ID NO: 6.

In another aspect of the present invention, a dough is presented comprising a phospholipase A1 enzyme characterized by having an sn1/sn2 specificity ratio of about 55/45 or greater wherein the phospholipase A1 has a lysophospholipase/phospholipase activity ratio of less than 0.01. Preferably, the dough has improved extensibility and/or stability. In another aspect of the present invention, the dough further has at least one additional enzyme selected from the group consisting of amylase, cyclodextrin glucanotransferase, peptidase, transglutaminase, lipase, galactolipase, phospholipase which is different than the phospholipase A1, cellulase, hemicellulase, protease, protein disulfide isomerase, glycosyltransferase, peroxidase, lipoxygenase, laccase, and oxidase. Preferably, the amylase is an exoamylase. Preferably, the exoamylase is a maltogenic amylase. Preferably, the exoamylase is a non-maltogenic amylase. More preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. In another preferred embodiment, the additional enzyme is a phospholipase. More preferably, the phospholipase has galactolipase activity. In another preferred embodiment, the phospholipase is SEQ ID NO: 17 and/or SEQ ID NO: 18.

In another aspect of the present invention, a method of preparing a baked product is presented in which a dough as described above is baked. In another aspect of the present invention, a baked product is presented. Preferably, the baked product has at least one improved property selected from the group consisting of improved crumb pore size, improved uniformity of gas bubbles, no separation between crust and crumb, increased volume, increased crust crispiness and improved oven spring. More preferably, the improved property is increased crust crispiness.

In another aspect of the present invention, a pre-mix for baking is presented comprising flour and a phospholipase A1 enzyme characterized by having an sn1/sn2 specificity ratio of about 55/45 or greater wherein the phospholipase A1 has a lysophospholipase/phospholipase activity ratio of less than 0.01. In another aspect of the present invention, the pre-mix for baking has at least one additional enzyme selected from the group consisting of amylase, cyclodextrin glucanotransferase, peptidase, transglutaminase, lipase, galactolipase, phospholipase which is different than the phospholipase A1, cellulase, hemicellulase, protease, protein disulfide isomerase, glycosyltransferase, peroxidase, lipoxygenase, laccase, and oxidase. Preferably, the amylase is an exoamylase. Preferably, the exoamylase is a maltogenic amylase. Preferably, the exoamylase is a non-maltogenic amylase. More preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. More preferably, the phospholipase has galactolipase activity. In another preferred embodiment, the phospholipase is SEQ ID NO: 17 and/or SEQ ID NO: 18.

In another aspect of the present invention, a baking improver is presented comprising a granulate or agglomerated powder comprising a phospholipase A1 enzyme characterized by having an sn1/sn2 specificity ratio of about 55/45 or greater wherein the phospholipase A1 has a lysophospholipase/phospholipase activity ratio of less than 0.01. In another aspect of the present invention, the baking improver has at least one additional enzyme selected from the group consisting of amylase, cyclodextrin glucanotransferase, peptidase, transglutaminase, lipase, galactolipase, phospholipase which is different than the phospholipase A1, cellulase, hemicellulase, protease, protein disulfide isomerase, glycosyltransferase, peroxidase, lipoxygenase, laccase, and oxidase. Preferably, the amylase is an exoamylase. Preferably, the exoamylase is a maltogenic amylase. Preferably, the exoamylase is a non-maltogenic amylase. More preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. More preferably, the phospholipase has galactolipase activity. In another preferred embodiment, the phospholipase is SEQ ID NO: 17 and/or SEQ ID NO: 18.

In another aspect of the present invention, a method of making a dough is presented as set forth above but in which at least one additional enzyme useful for improving dough and/or a baked product made therefrom is included. Preferably, the additional enzyme is selected from the group consisting of amylase, cyclodextrin glucanotransferase, peptidase, transglutaminase, lipase, galactolipase, phospholipase which is different than the phospholipase A1, cellulase, hemicellulase, protease, protein disulfide isomerase, glycosyltransferase, peroxidase, lipoxygenase, laccase, and oxidase. Preferably, the amylase is an exoamylase. Preferably, the exoamylase is a maltogenic amylase. Preferably, the exoamylase is a non-maltogenic amylase. More preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. More preferably, the phospholipase has galactolipase activity. In another preferred embodiment, the phospholipase is SEQ ID NO: 17 and/or SEQ ID NO: 18.

In another aspect of the present invention, a method for modification of a phospholipid emulsifier comprising treatment of the emulsifier with a phospholipase A1 enzyme characterized by having an sn1/sn2 specificity ratio of about 55/45 or greater wherein the phospholipase A1 has a lysophospholipase/phospholipase activity ratio of less than 0.01. Optionally, the emulsifier is lecithin.

In another aspect of the present invention, a method of creating a lysophospholipid in a lipid containing food matrix is presented comprising adding to the lipid containing food matrix a phospholipase A1 enzyme characterized by having an sn1/sn2 specificity ratio of about 55/45 or greater wherein the phospholipase A1 has a lysophospholipase/phospholipase activity ratio of less than 0.01. Preferably, the lipid containing food matrix is selected from the group consisting of eggs and food products containing eggs such as dough for sweet bakery goods, processed meat, milk based products, vegetable oil and sweet bakery goods, including cakes and cookies.

### Assays and Methods

### Enzyme Characterization Assays—Activity Assays and Assay for the Determination of Phospholipase Position Specificity

### PC-P Assay:

Phospholipase activity (PC-U) may be determined using the following assay:
Substrate: 1.71% L-α-phosphatidylcholine Soy (95%) (Avanti 441601G, Avanti Polare Lipids, USA), 6.25% TRITON™-X 100 (Sigma X-100), and 5 mM CaCl₂ were dissolved in 0.05 M HEPES buffer pH 7.

### Assay Procedure:

Samples, calibration sample, and control sample were diluted in 10 mM HEPES pH 7.0 containing 0.1% TRITON™ X-100. Analysis was carried out using 96 well microtiter plate and a ThermoMixcer C (Eppendorf, Germany). The assay was run at 30° C. 200 μL substrate was thermostated for 180 seconds at 30° C., before 50 μL of enzyme sample was added. Enzymation lasted 600 sec. The amount of free fatty acid liberated during enzymation was measured using the NEFA kit obtained from WakoChemicals GmbH, Germany).
This assay kit is composed of two reagents

### NEFA-HR(1):

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR(2):

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)
   After incubation 10 μl enzymation mixture was transferred to a new micro titer plate containing 150 μL NEFA-HR(1) and incubated for 240 sec at 30° C. Afterwards 75 μL NEFA-HR(2) was added and the mixture was incubated for 240 sec at 30° C. OD 540 nm was then measured.
   Enzyme activity (μmol FFA/(min·mL)) was calculated based on a calibration curve made form oleic acid. Enzyme activity PC-U was calculated as micromole fatty acid produced per milliliter volume of enzyme sample per minute under assay conditions.

$$Enzyme activity ( μmol / ( min \cdot mL ) ) = \frac{OD * 250 μ1 * D}{S * 50 μ1 * 10 min}$$

$$OD = OD of sample withdrawn OD of blind sample$$

$$250 μ1 = total volume of substrate and enzyme$$

$$50 μ1 = Volume of enzyme solution$$

$$D = dilution of sample$$

$$S = the slope of the calibration curve ( OD / ( μmol / mL ) )$$

$$10 = reaction time of enzymation ( min )$$

### LPC-P Assay:

Lyso-Phospholipase activity (LPC-U) may be determined using the following assay:
Substrate: 1.18% 1-oleoyl-2-hydroxy-sn-glycero-3-phosphocholine (Avanti 845875P, Avanti Polar lipid, USA), 6.25% TRITON™-X 100 (Sigma X-100), and 5 mM CaCl₂ were dissolved in 0.05 M HEPES buffer pH 7.

### Assay Procedure:

Samples, calibration sample, and control sample were diluted in 10 mM HEPES pH 7.0 containing 0.1% TRITON™ X-100. Analysis was carried out using 96 well micro titer plate and a ThermoMixcer C (Eppendorf, Germany). The assay was run at 30° C. 200 μL substrate was thermostated for 180 seconds at 30° C., before 50 μL of enzyme sample was added. Enzymation lasted 600 sec. The amount of free fatty acid liberated during enzymation was measured using the NEFA kit obtained from WakoChemicals GmbH, Germany).
This assay kit is composed of two reagents

### NEFA-HR(1):

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR(2):

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)
   After incubation 10 μl enzymation mixture was transferred to a new micro titer plate containing 150 μL NEFA-HR(1) and incubated for 240 sec at 30° C. Afterwards 75 μL NEFA-HR(2) was added and the mixture was incubated for 240 sec at 30° C. OD 540 nm was then measured.
   Enzyme activity (μmol FFA/(min·mL)) was calculated based on a calibration curve made form oleic acid. Enzyme activity LPC-U was calculated as micromole fatty acid produced per milliliter volume of enzyme sample per minute under assay conditions.

$$Enzyme activity ( μmol / ( min \cdot mL ) ) = \frac{OD * 250 μ1 * D}{S * 50 μ1 * 10 min}$$

$$OD = OD of sample withdrawn OD of blind sample$$

$$250 μ1 = total volume of substrate and enzyme$$

$$50 μ1 = Volume of enzyme solution$$

$$D = dilution of sample$$

$$S = the slope of the calibration curve ( OD / ( μmol / mL ) )$$

$$10 = reaction time of enzymation ( min )$$

### NAPE-P Assay:

NAPE Phospholipase activity (NAPE-U) may be determined using the following assay:
Substrate: 2.25% Palmitoyl-2-linoleoyl-sn-glycero-3-phosphoethanolamine-N-linoleoyl (16:0-18:2 PE-N18:2) (Avanti 792003, Avanti Polar lipid, USA), 6.25% TRITON™-X 100 (Sigma X-100), and 5 mM CaCl₂ were dissolved in 0.05 M HEPES buffer pH 7.

### Assay Procedure:

Samples, calibration sample, and control sample were diluted in 10 mM HEPES pH 7.0 containing 0.1% TRITON™ X-100. Analysis was carried out using 96 well micro titer plate and a ThermoMixcer C (Eppendorf, Germany). The assay was run at 30° C. 200 μL substrate was thermostated for 180 seconds at 30° C., before 50 μL of enzyme sample was added.
Enzymation lasted 600 sec. The amount of free fatty acid liberated during enzymation was measured using the NEFA kit obtained from WakoChemicals GmbH, Germany).
This assay kit is composed of two reagents

### NEFA-HR(1):

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR(2):

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)

After incubation 10 μl enzymation mixture was transferred to a new micro titer plate containing 150 μL NEFA-HR(1) and incubated for 240 sec at 30° C. Afterwards 75 μL NEFA-HR(2) was added and the mixture was incubated for 240 sec at 30° C. OD 540 nm was then measured.

Enzyme activity (μmol FFA/min·mL) was calculated based on a calibration curve made form oleic acid. Enzyme activity NAPE-U pH 7 was calculated as micromole fatty acid produced per minute under assay conditions.

Enzyme activity (μmol FFA/(min·mL)) was calculated based on a calibration curve made form oleic acid. Enzyme activity NAPE-U was calculated as micromole fatty acid produced per milliliter volume of enzyme sample per minute under assay conditions.

$$Enzyme activity ( μmol / ( min \cdot mL ) ) = \frac{OD * 250 μ1 * D}{S * 50 μ1 * 10 min}$$

$$OD = OD of sample withdrawn OD of blind sample$$

$$250 μ1 = total volume of substrate and enzyme$$

$$50 μ1 = Volume of enzyme solution$$

$$D = dilution of sample$$

$$S = the slope of the calibration curve ( OD / ( μmol / mL ) )$$

$$10 = reaction time of enzymation ( min )$$

### NALPE-P Assay:

NALPE Phospholipase activity (NALPE-U) may be determined using the following assay:
Substrate: 1.68% 1-palmitoyl-sn-glycero-3-phosphoethanolamine-N-linoleoyl (16:0-NALPE-N18:2), (Avanti 791759, Avanti Polar Lipids, USA), 6.25% TRITON™-X 100 (Sigma X-100), and 5 mM CaCl₂ were dissolved in 0.05 M HEPES buffer pH 7.

### Assay Procedure:

Samples, calibration sample, and control sample were diluted in 10 mM HEPES pH 7.0 containing 0.1% TRITON™ X-100. Analysis was carried out using 96 well micro titer plate and a ThermoMixcer C (Eppendorf, Germany). The assay was run at 30° C. 200 μL substrate was thermostated for 180 seconds at 30° C., before 50 μL of enzyme sample was added. Enzymation lasted 600 sec. The amount of free fatty acid liberated during enzymation was measured using the NEFA kit obtained from WakoChemicals GmbH, Germany).
This assay kit is composed of two reagents

### NEFA-HR(1):

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR(2):

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)
   After incubation 10 μl enzymation mixture was transferred to a new micro titer plate containing 150 μL NEFA-HR(1) and incubated for 240 sec at 30° C. Afterwards 75 μL NEFA-HR(2) was added and the mixture was incubated for 240 sec at 30° C. OD 540 nm was then measured.
   Enzyme activity (μmol FFA/(min·mL)) was calculated based on a calibration curve made form oleic acid. Enzyme activity NALPE-U was calculated as micromole fatty acid produced per milliliter volume of enzyme sample per minute under assay conditions.

$$Enzyme activity ( μmol / ( min \cdot mL ) ) = \frac{OD * 250 μ1 * D}{S * 50 μ1 * 10 min}$$

$$OD = OD of sample withdrawn OD of blind sample$$

$$250 μ1 = total volume of substrate and enzyme$$

$$50 μ1 = Volume of enzyme solution$$

$$D = dilution of sample$$

$$S = the slope of the calibration curve ( OD / ( μmol / mL ) )$$

$$10 = reaction time of enzymation ( min )$$

### Assay for the Determination of Phospholipase Activity and sn1 and sn2 Position Specificity on PC (Phosphatidylcholine)

Substrate: 0.6% 16:0-18:1 PC, 1-palmitoyl-2-oleoyl-sn-glycero-3-phosphocholine (Avanti 850457, Avanti Polar Lipids, USA), 0.4% TRITON™-X 100 (Sigma, X-100), and 5 mM CaCl₂ were dissolved in 0.05 M HEPES buffer pH 7.

### Assay Procedure:

2 mL substrate was incubated at 30° C. and added 0.1 ml of an enzyme dilution corresponding to 2-10% substrate consumed after 10 minutes reaction in 0.05 M HEPES buffer (magnetic stirring).
40 μL 4 M HCl was added to stop the reaction and to protonate the free fatty acids. 1 mL 99% ethanol was added and mixed on a Vortex mixer. 5 mL MTBE (methyl tert-butyl ether) containing 0.5 mg C17:0 fatty acid (margaric acid) was added. The sample was mixed again on a Vortex mixer for 5 sec and extracted for 30 minutes on a Rotamix at 25 rpm. The sample was centrifuged at 1520 g for 10 minutes.
One 500 mg amine (NH₂)—Bond Elut SPE column (Agilent) was placed on a Bond Elut Vacuum System. The column was conditioned with 8 mL Petroleum-ether. The MTBE phase from the extraction was applied onto the column and eluted with:
1. fraction 8 mL Solvent A: MTBE:2-propanol (2:1)
2. fraction 8 mL Solvent B: Acetone:Formic acid (100:2)
   The solvents were eluted with approx. 0.25 mL/min.
   The collected fatty acid fraction (fract. 2) was evaporated to dryness and fatty acids were analyzed by GLC. Based on the internal standard Fatty Acid C17:0 the amount of C16:0 and C18:1 fatty acid was determined.
   Enzyme activity was calculated as μmol fatty acid produced per minutes under assay conditions.

$$Enzyme activity = \frac{2 \times A \times 1000000 \times D}{100 \times MW \times 10 \times 0.1}$$

$$Where$$

$$A = \% C 16 : 0 fatty acid + \% C 18 : 1 fatty acids$$

$$2 = mL substrate$$

$$1000000 = mol conversion to μmol$$

$$D = Enzyme dilution factor$$

$$MW = average molecular weight of C 16 : 0 and C 18 : 1 fatty acids produced$$

$$10 = minutes reaction time$$

$$0.1 = mL enzyme added to assay$$

The relative PLA1 enzyme activity was calculated as:

$$Relative PLA 1 activity = \frac{\% C 16 : 0 \times 100}{\% C 16 : 0 + \% C 18 : 1}$$

The relative PLA2 enzyme activity was calculated as:

$$Relative PLA 2 activity = \frac{\% C 18 : 1 \times 100}{\% C 16 : 0 + \% C 18 : 1}$$

The sn1/sn2 specificity ratio is presented as:

Sn1/sn2 specificity ratio=Relative PLA1 activity/Relative PLA2 activity

### Gaschromatography (GLC):

Free fatty acid was analyzed by GLC as trimethyl silyl derivatives (TMS).

### Apparatus

Perkin Elmer Clarus 600 Capillary Gas Chromatograph equipped with WCOT fused silica column 12.5 m×0.25 mm ID×0.1 μ film thickness 5% phenyl-methyl-silicone (CP Sil 8 CB from Chrompack).
Carrier gas: Helium.
Injector: PSSI cold split injection (initial temp 90° C. heated to 395° C.), volume 1.0 μl
Detector FID: 395° C.

| Oven program: | 1 | 2 | 3 | 4 |
|---|---|---|---|---|
| Oven temperature, ° C. | 80 | 200 | 240 | 360 |
| Isothermal, time, min | 2 | 0 | 0 | 10 |
| Temperature rate, ° C./min | | 20 | 10 | 12 |

### Sample Preparation:

Evaporated sample is dissolved in 1.5 ml Heptane:Pyridine, 2:1. 500 μl sample solution is transferred to a crimp vial, 100 μl MSTFA (N-Methyl-N-trimethylsilyl-trifluoraceamid) is added and reacted for 15 minutes at 60° C.

### Baking Applications

Crusty Roll baking setup

| Recipe | Bakers % |
|---|---|
| Wheat flour (Reform) | 100 |
| Compressed yeast (Malteserkors) | 4.5 |
| Salt | 1.6 |
| Sugar | 1.6 |
| Water (400 BU-2%) | 57 |
| Fungal alpha amylase (16.2 FAU/g | 0.46 |
| blend) | |
| Other Enzymes | variable |

Kneading on a Diosna spiral mixer. Water uptake for flour according to analysis: 400 BU-2%

### Procedure

Mix all ingredients in a bowl, 1 minute slow speed—add water and knead 2 minutes slow and 6.5 minutes fast speed. Dough temperature must be approximate 26° C. 1350 g dough is scaled and molded round by hand. The dough is rested in a heating cabinet for 10 minutes at 30° C.
The dough is molded into 30 dough balls on a “GLIMIK™ rounder”—settings according to table on machine.
The dough is proofed for 45 minutes at 34° C., 85% RH and baked for 13 minutes at 200° C. / 2 l steam+5 minutes damper open (MIWE oven prog. 1). After baking the rolls are cooled for 25 minutes at ambient temperature before weighing and measuring of volume.
Dough and bread characteristics are evaluated by a skilled person

| | | | |
|---|---|---|---|
| Evaluation | Evaluation method | Lowest score = 1 | Highest score = 10 |

Dough

| | | | |
|---|---|---|---|
| Dough | Extend dough | Dough cannot be | Dough can be |
| development | with fingers | stretched without | stretched obtaining |
| after mixing | | breaking | papery thin dough |
| | | | without breakage |
| Stickiness | Cut a big slit in | Dry surface. The | The dough sticks |
| after mixing | all dough, open | dough slips your | to your fingers |
| | the dough, touch | fingers | |
| | the cut dough | | |
| | surface with | | |
| | fingers | | |
| Extensibility | Extend dough | Dough cannot be | Dough can be |
| after resting | with fingers | stretched without | stretched obtaining |
| | | breaking | papery thin dough |
| | | | without breakage |
| Stickiness | Cut a big slit in | Dry surface. The | The dough sticks |
| after resting | all dough, open | dough slips your | to your fingers |
| | the dough, touch | fingers | |
| | the cut dough | | |
| | surface with | | |
| | fingers | | |

Crust

| | | | |
|---|---|---|---|
| Crispiness of | Fracture crust | Leathery crust | Crisp crust |
| crust | using several | | |
| | fingers | | |

Crumb

| | | | |
|---|---|---|---|
| Crumb pore | Visual evaluation | Open crumb, big | Fine crumb, small |
| size | of sliced bread, | gas bubbles | gas bubbles |
| | size of gas | | |
| | bubbles in crumb | | |
| Crumb pore | Visual evaluation | Big variation in | Constant gas |
| homogeneity | of sliced bread, | sizes of gas | bubble size |
| | homogeneity of | bubbles | |
| | gas bubbles | | |

Product shape

| | | | |
|---|---|---|---|
| Capping/ | Visual evaluation | A very large | No separation |
| Hole under the | of vertical cut | hole directly | between crust and |
| crust | surface | under the crust. | crumb. |
| Oven | Visual evaluation | No energy | High level of |
| spring/Energy | amount of energy | | energy |
| | in the product | | |

### Extraction of Dough Lipids.

Sample of fully proofed dough was frozen and freeze dried. The dry dough was the grounded and sieved. 1.5 g grounded, sifted sample was mixed with 1.5 g carrier (Diatomaceous earth, Thermo Scientific, P/N:60-033854) and transferred into a ASE 10 ml sample tube. Extraction was carried out using Dionex ASE350 (Thermo Scientific) at 40° C. with water saturated butanol as solvent and a static run time of 10 minutes. After extraction, the solvent was evaporated using Scan Speed 40 (Scanvac, Labogene APS) at 60° C. and 1000 rpm. The dried lipid was dissolved in 3.75 ml Heptane:Isopropanol (3:2).

HPLC Analysis of Phospholipids Extracted from Dough:

The dough lipid samples were analyzed by liquid chromatography using a Charged Aerosol Detector. The column was a normal phase column (DIOL) and the mobile phase was a gradient of A: acetone/methanol 96/4 with addition of 1 mM ammonium formate and B: acetone/methanol/H₂O 60/34/6 with addition of 1 mM ammonium formate.

NALPE was used as standard for quantification.

### Instrumental:

### Dionex Ultimate 3000 UHPLC, Thermo Scientific

### VANQOISH Detector, Thermo Scientific

### Column: Fortis HILIC Diol, 1.7 μm, 50×2.1 mm

Chromatographic:

| Time (min) | Mobile phase A | Mobile phase B |
|---|---|---|
| 0 | 100% | 0% |
| 20 | 0% | 100% |
| 30 | 0% | 100% |

Column temperature was 30° C. and injection volume was 4 μL.

### Sample Preparation:

Lipid was extracted from dough as described in ‘Extraction of dough lipids’ and filtered through 0.45 μM filter before being injected.

### Calculation:

Cromeleon software was used to integrate the chromatograms and molar concentration of NAPE, NALPE and NAGPE was calculated based on a NALPE standard curve.

### Presentation of Results:

Respective lipid levels of NAPE, NALPE and NAGPE were obtained by initially normalizing the respective molar level of each component to the ‘Average Total molar lipid (NAPE+NALPE+NAGPE)’ across all doughs. Following, respective lipid levels are presented relative to NAPE level in the Negative control (no enzyme added). Thus, NAPE starts (Negative control) at 1. NALPE and NAGPE are presented as levels generated relative to NAPE start level.

### Chemical Structures

In below structures R1, R2 and R3 are C12-C24 hydrocarbons. The C12-24 hydrocarbons are either saturated or unsaturated. R1, R2 and R3 may be identical or different hydrocarbons.

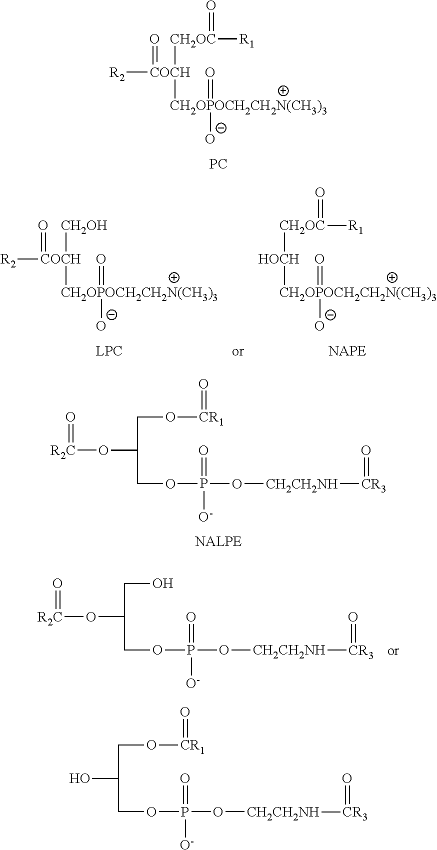

It should be kept in mind that the following described embodiment(s) is only presented by way of example and should not be construed as limiting the inventive concept to any particular enzyme.

### EXAMPLES

### Example 1—CRC08310—ThaPla1

### Cloning of Trichoderma harzianum Phospholipase ThaPla1 (CRC08310)

A putative phospholipase gene, designated as CRC08310, was identified in Trichoderma harzianum and encodes a protein with 100% homology to a sequence available from the NCBI database (NCBI accession No.: KKO98756.1) as determined from a BLAST search (Altschul et al., J Mol Biol, 215: 403-410, 1990). The codon-optimized synthetic nucleic acid sequence of full-length CRC08310 is provided in SEQ ID NO: 19. The corresponding protein encoded by the full-length CRC08310 gene is shown in SEQ ID NO:1. At the N-terminus, the protein has a signal peptide with a length of 16 amino acids as predicted by SignalP version 4.0 (Nordahl Petersen et al. (2011) Nature Methods, 8:785-786). The presence of a signal sequence suggests that CRC08310 is a secreted enzyme. The predicted, mature protein sequence of CRC08310 is set forth in SEQ ID NO: 2.

### Example 2—Expression of CRC08310

The codon-optimized synthetic DNA sequence encoding the full-length CRC08310 protein (SEQ ID NO: 19) was synthesized and inserted into the Trichoderma reesei expression vector pGXT (the same as the pTTTpyr2 vector described in published PCT Application WO2015/017256, incorporated by reference herein), resulting in plasmid pGXT-CRC08310. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08310 contains the Trichoderma reesei cbhI-derived promoter (cbhI) and cbhI terminator regions allowing for a strong inducible expression of the gene of interest.

The pGXT-CRC08310 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

### Example 3—CRC08316—PfiPla1

### Cloning of Pestalotiopsis fici W106-1 Phospholipase PfiPla1 (CRC08316)

A putative phospholipase gene, designated as CRC08316, was identified in Pestalotiopsis fici W106-1 and encodes a protein with 100% homology to a sequence available from the NCBI database (NCBI accession No.: ETS81250.1) as determined from a BLAST search (Altschul et al., J Mol Biol, 215: 403-410, 1990). The codon-optimized synthetic nucleic acid sequence of full-length CRC08316 is provided in SEQ ID NO: 20. The corresponding protein encoded by the full-length CRC08316 gene is shown in SEQ ID NO:3. At the N-terminus, the protein has a signal peptide with a length of 18 amino acids as predicted by SignalP version 4.0 (Nordahl Petersen et al. (2011) Nature Methods, 8:785-786). The presence of a signal sequence suggests that CRC08316 is a secreted enzyme. The predicted, mature protein sequence of CRC08316 is set forth in SEQ ID NO: 4.

### Example 4—Expression of CRC08316

The codon-optimized synthetic DNA sequence encoding the full-length CRC08316 protein (SEQ ID NO: 20) was synthesized and inserted into the Trichoderma reesei expression vector pGXT (the same as the pTTTpyr2 vector described in published PCT Application WO2015/017256, incorporated by reference herein), resulting in plasmid pGXT-CRC08316. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08316 contains the Trichoderma reesei cbhI-derived promoter (cbhI) and cbhI terminator regions allowing for a strong inducible expression of the gene of interest. The pGXT-CRC08316 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a 60 mL Phenyl-FF Sepharose column pre-equilibrated with the loading buffer containing 20 mM sodium acetate (pH 5.0) and 1 M ammonium sulfate. The target protein was eluted from the column with 20 mM sodium acetage (pH 5.0) and a gradient of 0.5-0.3 M ammonium sulfate. The fractions containing the active target protein were pooled, concentrated and subsequently loaded onto a HiLoad Q_HP Sepharose column pre-equilibrated with 20 mM Tris buffer (pH 8.0). The target protein was eluted from the column with 20 mM Tris buffer (pH 8.0) and a NaCl gradient of 0-0.4 M. The fractions containing the active target protein were then pooled and concentrated via the 10K Amicon Ultra devices, and stored in 20 mM Tris buffer (pH 8.0) and 40% glycerol at −20° C. until usage.

### Example 5—CRC08319—MguPla1

### Cloning of Metarhizium guizhouense ARSEF 977 Phospholipase MguPla1 (CRC08319)

A putative phospholipase gene, designated as CRC08319, was identified in Metarhizium guizhouense ARSEF 977 and encodes a protein with 100% homology to a sequence available from the NCBI database (NCBI accession No.: KID92477.1) as determined from a BLAST search (Altschul et al., J Mol Biol, 215: 403-410, 1990). The codon-optimized synthetic nucleic acid sequence of full-length CRC08319 is provided in SEQ ID NO: 21. The corresponding protein encoded by the full-length CRC08319 gene is shown in SEQ ID NO: 5. At the N-terminus, the protein has a signal peptide with a length of 16 amino acids as predicted by SignalP version 4.0 (Nordahl Petersen et al. (2011) Nature Methods, 8:785-786). The presence of a signal sequence suggests that CRC08319 is a secreted enzyme. The predicted, mature protein sequence of CRC08319 is set forth in SEQ ID NO: 6.

### Example 6—Expression of CRC08319

The codon-optimized synthetic DNA sequence encoding the full-length CRC08319 protein (SEQ ID NO: 21) was synthesized and inserted into the Trichoderma reesei expression vector pGXT (the same as the pTTTpyr2 vector described in published PCT Application WO2015/017256, incorporated by reference herein), resulting in plasmid pGXT-CRC08319. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08319 contains the Trichoderma reesei cbhI-derived promoter (cbhI) and cbhI terminator regions allowing for a strong inducible expression of the gene of interest.

The pGXT-CRC08319 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a 60 mL Phenyl-FF Sepharose column pre-equilibrated with the loading buffer containing 20 mM sodium phosphate (pH 7.0) and 1 M ammonium sulfate. The target protein was eluted from the column with 20 mM sodium phosphate (pH 7.0) and 0.25 M ammonium sulfate. The fractions containing the active target protein were pooled, concentrated and subsequently loaded onto a Superdex 75 gel filtration column pre-equilibrated with 20 mM sodium phosphate buffer (pH 7.0) supplemented with additional 0.15 M NaCl and 10% glycerol. The fractions containing the active target protein were then pooled and concentrated via the 10K Amicon Ultra devices, and stored in 20 mM sodium phosphate buffer (pH 7.0) supplemented with 0.15 M NaCl and 40% glycerol at −20° C. until usage.

### Example 7—CRC08405—DamPla1

### Cloning of Diaporthe ampelina Phospholipase DamPla1 (CRC08405)

A putative phospholipase gene, designated as CRC08405, was identified in Diaporthe ampelina and encodes a protein with 100% homology to a sequence available from the NCBI database (NCBI accession No.: KKY36548.1) as determined from a BLAST search (Altschul et al., J Mol Biol, 215: 403-410, 1990). The codon-optimized synthetic nucleic acid sequence of full-length CRC08405 is provided in SEQ ID NO: 22. The corresponding protein encoded by the full-length CRC08405 gene is shown in SEQ ID NO: 7. At the N-terminus, the protein has a signal peptide with a length of 18 amino acids as predicted by SignalP version 4.0 (Nordahl Petersen et al. (2011) Nature Methods, 8:785-786). The presence of a signal sequence suggests that CRC08405 is a secreted enzyme. The predicted, mature protein sequence of CRC08405 is set forth in SEQ ID NO: 8.

### Example 8—Expression of CRC08405

The codon-optimized synthetic DNA sequence encoding the full-length CRC08405 protein (SEQ ID NO: 22) was synthesized and inserted into the Trichoderma reesei expression vector pGXT (the same as the pTTTpyr2 vector described in published PCT Application WO2015/017256, incorporated by reference herein), resulting in plasmid pGXT-CRC08405. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08405 contains the Trichoderma reesei cbhI-derived promoter (cbhI) and cbhI terminator regions allowing for a strong inducible expression of the gene of interest.

The pGXT-CRC08405 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a 60 mL Phenyl-FF Sepharose column pre-equilibrated with the loading buffer containing 20 mM sodium phosphate (pH 7.0) and 1 M ammonium sulfate. The target protein was eluted from the column with 20 mM sodium phosphate (pH 7.0). The fractions containing the active target protein were pooled, concentrated and subsequently loaded onto a HiPrep Q-XL Sepharose column pre-equilibrated with 20 mM Tris buffer (pH 8.0). The target protein was eluted with 20 mM Tris buffer (pH 8.0) and a NaCl gradient of 0-0.5 M. The fractions containing the active target protein were then pooled and concentrated via the 10K Amicon Ultra devices, and stored in 20 mM Tris buffer (pH 8.0) supplemented with 0.15 M NaCl and 40% glycerol at −20° C. until usage.

### Example 9—CRC08418—MorPla3

### Cloning of Magnaporthe oryzae Y34 Phospholipase MorPla3 (CRC08418)

A putative phospholipase gene, designated as CRC08418, was identified in Magnaporthe oryzae Y34 and encodes a protein with 100% homology to a sequence available from the NCBI database (NCBI accession No.: ELQ41978.1) as determined from a BLAST search (Altschul et al., J Mol Biol, 215: 403-410, 1990). The codon-optimized synthetic nucleic acid sequence of full-length CRC08418 is provided in SEQ ID NO: 23. The corresponding protein encoded by the full-length CRC08418 gene is shown in SEQ ID NO: 9. At the N-terminus, the protein has a signal peptide with a length of 25 amino acids as predicted by SignalP version 4.0 (Nordahl Petersen et al. (2011) Nature Methods, 8:785-786). The presence of a signal sequence suggests that CRC08418 is a secreted enzyme. The predicted, mature protein sequence of CRC08418 is set forth in SEQ ID NO: 10.

### Example 10—Expression of CRC08418

The codon-optimized synthetic DNA sequence encoding the full-length CRC08418 protein (SEQ ID NO: 23) was synthesized and inserted into the Trichoderma reesei expression vector pGXT (the same as the pTTTpyr2 vector described in published PCT Application WO2015/017256, incorporated by reference herein), resulting in plasmid pGXT-CRC0418. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08418 contains the Trichoderma reesei cbhI-derived promoter (cbhI) and cbhI terminator regions allowing for a strong inducible expression of the gene of interest.

The pGXT-CRC08418 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 0.8 M. After filtering, the resulting soluble fraction was applied to a 60 mL Phenyl-FF Sepharose column pre-equilibrated with the loading buffer containing 20 mM sodium phosphate (pH 7.0) and 1 M ammonium sulfate. The target protein was eluted from the column with 20 mM sodium phosphate (pH 7.0). The fractions containing the active target protein were pooled, concentrated and subsequently loaded onto a Superdex 75 gel filtration column pre-equilibrated with 20 mM sodium phosphate buffer (pH 7.0) with 0.15 M NaCl (pH 7.0). The fractions containing the active target protein were then pooled and concentrated via the 10K Amicon Ultra devices, and stored in 20 mM sodium phosphate buffer (pH 7.0) with 0.15 M NaCl (pH 7.0) and 40% glycerol at −20° C. until usage.

### Example 11—CRC08826—NdiPla1

### Cloning of Neonectria ditissima Phospholipase NdiPla1 (CRC08826)

A putative phospholipase gene, designated as CRC08826, was identified in Neonectria ditissima and encodes a protein with 100% homology to a sequence available from the NCBI database (NCBI accession No.: KPM45012.1) as determined from a BLAST search (Altschul et al., J Mol Biol, 215: 403-410, 1990). The codon-optimized synthetic nucleic acid sequence of full-length CRC08826 is provided in SEQ ID NO: 24. The corresponding protein encoded by the full-length CRC08826 gene is shown in SEQ ID NO: 11. At the N-terminus, the protein has a signal peptide with a length of 16 amino acids as predicted by SignalP version 4.0 (Nordahl Petersen et al. (2011) Nature Methods, 8:785-786). The presence of a signal sequence suggests that CRC08826 is a secreted enzyme. The predicted, mature protein sequence of CRC08826 is set forth in SEQ ID NO: 12.

### Example 12—Expression of CRC08826

The codon-optimized synthetic DNA sequence encoding the full-length CRC08826 protein (SEQ ID NO: 24) was synthesized and inserted into the Trichoderma reesei expression vector pGXT (the same as the pTTTpyr2 vector described in published PCT Application WO2015/017256, incorporated by reference herein), resulting in plasmid pGXT-CRC08826. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08826 contains the Trichoderma reesei cbhI-derived promoter (cbhI) and cbhI terminator regions allowing for a strong inducible expression of the gene of interest.

The pGXT-CRC08826 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a HiPrep Phenyl FF 16/10 column pre-equilibrated with the loading buffer containing 20 mM sodium phosphate (pH 5.0) and 1 M ammonium sulfate. The target protein was eluted from the column with 20 mM sodium phosphate (pH 5.0) and a gradient of 0.5-0 M ammonium sulfate. The fractions containing the active target protein were pooled, concentrated and subsequently loaded onto a HiPrep Q FF 16/10 column pre-equilibrated with 20 mM sodium phosphate buffer (pH 7.0). The target protein was eluted with 20 mM sodium phosphate buffer (pH 7.0) and a NaCl gradient of 0-0.5 M. The fractions containing the active target protein were then pooled, concentrated and subsequently loaded onto a HiLoad 26/60 Superdex 75 Prep column pre-equilibrated with 20 mM sodium acetate (pH 5.0) and 150 mM NaCl. The fractions containing the active target protein were pooled, concentrated and loaded onto a HiPrep Phenyl HP 16/10 column pre-equilibrated with the loading buffer containing 20 mM sodium phosphate (pH 5.0) and 1 M ammonium sulfate. The target protein was eluted with 20 mM sodium phosphate (pH 5.0) and a gradient of 0.75-0 M ammonium sulfate. The fractions containing the active target protein were pooled, concentrated via the 10K Amicon Ultra devices, and stored in 20 mM sodium phosphate (pH 5.0) and 40% glycerol at −20° C. until usage.

### Example 13—CRC08833—TgaPla1

### Cloning of Trichoderma gamsii Phospholipase TgaPla1 (CRC08833)

A putative phospholipase gene, designated as CRC08833, was identified in Trichoderma gamsii and encodes a protein with 100% homology to a sequence available from the NCBI database (NCBI accession No.: KUF04745.1) as determined from a BLAST search (Altschul et al., J Mol Biol, 215: 403-410, 1990). The codon-optimized synthetic nucleic acid sequence of full-length CRC08833 is provided in SEQ ID NO: 25. The corresponding protein encoded by the full-length CRC08833 gene is shown in SEQ ID NO: 13. At the N-terminus, the protein has a signal peptide with a length of 16 amino acids as predicted by SignalP version 4.0 (Nordahl Petersen et al. (2011) Nature Methods, 8:785-786). The presence of a signal sequence suggests that CRC08833 is a secreted enzyme. The predicted, mature protein sequence of CRC08826 is set forth in SEQ ID NO: 14.

### Example 14—Expression of CRC08833

The codon-optimized synthetic DNA sequence encoding the full-length CRC08833 protein (SEQ ID NO: 25) was synthesized and inserted into the Trichoderma reesei expression vector pGXT (the same as the pTTTpyr2 vector described in published PCT Application WO2015/017256, incorporated by reference herein), resulting in plasmid pGXT-CRC08833. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08833 contains the Trichoderma reesei cbhI-derived promoter (cbhI) and cbhI terminator regions allowing for a strong inducible expression of the gene of interest.

The pGXT-CRC08833 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a 60 mL Phenyl-FF Sepharose column pre-equilibrated with the loading buffer containing 20 mM sodium phosphate (pH 7.0) and 1 M ammonium sulfate. The target protein was eluted from the column with 20 mM sodium phosphate (pH 7.0) and 0.5 M ammonium sulfate. The fractions containing the active target protein were pooled, concentrated and subsequently loaded onto a HiLoad Q_XL Sepharose column pre-equilibrated with 20 mM Tris buffer (pH 8.0). The target protein was eluted with 20 mM Tris buffer (pH 8.0) and a NaCl gradient of 0-0.5 M. The fractions containing the active target protein were then pooled and concentrated via the 10K Amicon Ultra devices, and stored in 20 mM Tris buffer (pH 8.0) and 40% glycerol at −20° C. until usage.

### Example 15—CRC08845—ManPla1

### Cloning of Metarhizium anisopliae BRIP 53293 Phospholipase ManPla1 (CRC08845)

A putative phospholipase gene, designated as CRC08845, was identified in Metarhizium anisopliae BRIP 53293 and encodes a protein with 100% homology to a sequence available from the NCBI database (NCBI accession No.: KJK84204.1) as determined from a BLAST search (Altschul et al., J Mol Biol, 215: 403-410, 1990). The codon-optimized synthetic nucleic acid sequence of full-length CRC08845 is provided in SEQ ID NO: 26. The corresponding protein encoded by the full-length CRC08845 gene is shown in SEQ ID NO: 15. At the N-terminus, the protein has a signal peptide with a length of 17 amino acids as predicted by SignalP version 4.0 (Nordahl Petersen et al. (2011) Nature Methods, 8:785-786). The presence of a signal sequence suggests that CRC08845 is a secreted enzyme. The predicted, mature protein sequence of CRC08845 is set forth in SEQ ID NO: 16.

### Example 16—Expression of CRC08845

The codon-optimized synthetic DNA sequence encoding the full-length CRC08845 protein (SEQ ID NO: 26) was synthesized and inserted into the Trichoderma reesei expression vector pGXT (the same as the pTTTpyr2 vector described in published PCT Application WO2015/017256, incorporated by reference herein), resulting in plasmid pGXT-CRC08845. In the pGXT vector, the Aspergillus nidulans pyrG gene is replaced with the Trichoderma reesei pyr2 gene. The Aspergillus nidulans amdS and pyr2 selective markers confer growth of transformants on acetamide as a sole nitrogen source, and the Trichoderma reesei telomere regions allow for non-chromosomal plasmid maintenance in a fungal cell. pGXT-CRC08845 contains the Trichoderma reesei cbhI-derived promoter (cbhI) and cbhI terminator regions allowing for a strong inducible expression of the gene of interest.

The pGXT-CRC08845 plasmid was then transformed into a suitable Trichoderma reesei strain (method described in published PCT application WO 05/001036) using protoplast transformation (Te'o et al. (2002) J. Microbiol. Methods 51:393-99). Transformants were selected on a solid medium containing acetamide as the sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies appeared in about 1 week. After growth on acetamide plates, transformants were picked and transferred individually to acetamide agar plates. After 5 days of growth on acetamide plates, transformants displaying stable morphology were inoculated in 200 μL glucose/sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250-mL shake flask with Glucose/Sophorose defined media.

The crude broth was concentrated to about 80 mL using a VivaFlow 200 ultrafiltration device (Sartorius Stedim). Ammonium sulfate was then added to the concentrated solution to a final concentration of 1 M. After filtering, the resulting soluble fraction was applied to a Butyl FF column pre-equilibrated with the loading buffer containing 20 mM sodium acetate (pH 5.0) and 1 M ammonium sulfate. The target protein was eluted from the column with 20 mM sodium acetate (pH 5.0) and a gradient of 0.3-0 M ammonium sulfate. The fractions containing the active target protein were pooled, concentrated and subsequently loaded onto a Q HP column pre-equilibrated with 20 mM sodium phosphate buffer (pH 7.0). The target protein was eluted with 20 mM sodium phosphate buffer (pH 7.0) and a NaCl gradient of 0-0.5 M. The fractions containing the active target protein were then pooled, concentrated and subsequently loaded onto a Q HP column pre-equilibrated with 20 mM Tris buffer (pH 8.0). The target protein was eluted with 20 mM Tris buffer (pH 8.0) and a NaCl gradient of 0-0.5 M. The fractions containing the active target protein were then pooled, concentrated via the 10K Amicon Ultra devices, and stored in 20 mM Tris buffer (pH 8.0), 0.15 M NaCl and 40% glycerol at −20° C. until usage.

### Example 17. Characterization of Phospholipases of the Present Invention Relative to Powerbake 4080 and Lipopan F

Enzyme characterization is done by determination of specific activity using different lipid substrates as per activity methods presented in ‘Assays and Methods’. Powerbake 4080 is a a commercial product of DuPont. Powerbake 4080 acts on a polar lipid at the sn1 position. The active enzyme component of Powerbake 4080 is set forth as SEQ ID NO: 6 from U.S. Pat. No. 8,012,732 hereby incorporated by reference (also set forth herein as SEQ ID NO: 17). This enzyme is known to have both galactolipase and phospholipase activity. Lipopan F is a commercial product of Novozymes. The active enzyme in Lipopan F acts on polar lipid at the sn1position and is in SEQ ID NO: 2 of EP0869167B hereby incorporated by reference (also set forth herein as SEQ ID NO: 18). This enzyme is also known to have galactolipase activity.

Specific activities are determined using phosphatidylcholine substrate (PC-P assay), lyso-phosphatidylcholine substrate (LPC-P assay), N-acyl phosphatidylethanolamine substrate (NAPE-P assay) and lyso-N-acyl phosphatidylethanolcholine substrate (NALPE-P assay). Activities are presented relative to protein concentration, presenting the specific activity of the various enzymes using different substrates—see Table 2.

TABLE 2
Specific activities of enzyme (activity unit/mg enzyme protein)

| | PC-U/mg | LPC-U/mg | NAPE-U/mg | |
|---|---|---|---|---|
| | enzyme | enzyme | enzyme | NALPE-U/mg |
| CRC0 | protein | protein | protein | enzyme protein |
| 8319 | 298 | 0.22 | 1439 | 0.67 |
| 8405 | 154 | 0.08 | 28 | 0.02 |
| 8418 | 218 | 0.17 | 1192 | 0.55 |
| 8826 | 328 | 0.19 | 2008 | 0.32 |
| 8845 | 156 | 0.31 | 1357 | 0.54 |
| 8316 | 307 | 0.12 | 16 | 0.02 |
| 8310 | 448 | 0.25 | 1653 | 0.35 |
| 8833 | 40 | 0.07 | 7 | 0.01 |
| Powerbake 4080 | 1416 | 28 | 1591 | 317 |
| Lipopan F | 605 | 12.7 | 763 | 105 |

As can be seen from Table 2 all enzymes (except Powerbake 4080 and Lipopan F) show very low specific activity for LPC and NALPE substrate. The ratio of LPC to PC as well as ratio of NALPE to NAPE activity is presented in Table 3.

TABLE 3
Ratio of specific activity for LPC to PC (LPC-U/PC-U) and NALPE to
NAPE (NALPE-U/NAPE-U). More specifically LPC-U/PC-U = (LPC-
U/mg protein)/(PC-U/mg protein) and NALPE-U/NAPE-U = (NALPE-
U/mg protein)/(NAPE-U/mg protein).

| CRC0 | SEQ ID NO: | LPC-U/PC-U | NALPE-U/NAPE-U |
|---|---|---|---|
| 8319 | 6 | 0.00072 | 0.00047 |
| 8405 | 8 | 0.00054 | 0.00070 |
| 8418 | 10 | 0.00080 | 0.00046 |
| 8826 | 12 | 0.00059 | 0.00016 |
| 8845 | 16 | 0.00199 | 0.00040 |
| 8316 | 4 | 0.00040 | 0.00126 |
| 8310 | 2 | 0.00056 | 0.00021 |
| 8833 | 14 | 0.00174 | 0.00151 |
| Powerbake 4080 | 17 | 0.02009 | 0.19961 |
| Lipopan F | 18 | 0.02099 | 0.13745 |

It is clear from Table 3, that the candidates tested show significantly lower activity towards the lysophospholipid substrate relative to phospholipid substrates than the existing marketed enzyme products such as Powerbake 4080 and Lipopan F.

The candidates evaluated surprisingly represent a new group of phospholipases—‘No-lyso-phospholipases’—which are characterized by having No or extremely low lyso-phospholipase activity.

Current marketed products show LPC-U/PC-U or NALPE-U/NAPE-U ratios above respectively 0.014 and 0.13, whereas in contrast the ‘No-lyso-phospholipases’ show ratios below respectively, 0.002 and 0.0016. Thus, the ratios of the ‘No-lyso-phospholipases’ are lower than the current marketed products by a factor of 7 and 90, respectively.

This characteristic of ‘No-lyso-phospholipase’ activity provides the opportunity for a more robust system generating emulsifying components in lipid containing food matrix's. The ‘No-lyso-phospholipases’ provide more robust systems by elimination of the risk of over dosage as is seen with current marketed enzymes. The ‘No-lyso-phospholipases’ enable the generation of emulsifying components without risking the degradation of the generated emulsifying components (lyso-phospholipid like i.e. LPC or NALPE). Thus, the ‘roll-over effect’ observed with current marketed enzymes, where the lyso-phospholipid components are not only generated but also further hydrolyzed/degraded, is eliminated providing potential for overall higher levels of emulsifying components.

### Example 18. Characterization of Phospholipase Position Specificity

Enzyme position specificity was characterized by determination of free fatty acid (FFA) liberation from specific designed PC substrate. FFA determination was done by GLC analysis as presented in ‘Assay for the Determination of phospholipase activity and sn1 and sn2 position specificity on PC (phosphatidylcholine)’ under ‘Assays and Methods’.

The specificity was determined by assaying the release of free fatty acids (FFA) by GLC analysis. Based on the internal standard (Fatty Acid C17:0) the amount of C16:0 and C18:1 fatty acid was determined. Position specificity is presented as % relative PLA1 and % relative PLA2 activity. Please refer to Table 4 for specificity identification of the different candidates.

TABLE 4
Position specificity of Phospholipases as determined by ‘Assay
for the Determination of phospholipase activity and sn1 and
sn2 position specificity on PC (phosphatidylcholine)’ and
presentation of ratio sn1/sn2.

| | % Relative | | Ratio |
|---|---|---|---|
| PC (C16:0, C18:1) | PLA1 | PLA2 | sn1/sn2 |
| CRC07622 | 74 | 26 | 74/26 |
| CRC08310 | 72 | 28 | 72/28 |
| CRC08316 | 75 | 25 | 75/25 |
| CRC08319 | 74 | 26 | 74/26 |
| CRC08405 | 75 | 25 | 75/25 |
| CRC08418 | 99 | 1 | 99/1 |
| CRC08826 | 82 | 18 | 82/18 |
| CRC08833 | 75 | 25 | 75/25 |
| CRC08845 | 75 | 25 | 75/25 |
| Powerbake 4080 | 77 | 23 | 77/23 |
| Lipopan F | 75 | 25 | 75/25 |

### Example 19. Baking Experiments Testing Application Effect and Dough Lipid Profiling of ‘No-Lyso-Phospholipases’ vs Marketed Phospholipase Lipopan F

In this experiment, the current marketed phospholipase product Lipopan F was tested in a Crusty Roll experimental setup to show application performance by increasing dosages and the correlating lipid profiling of the dough matrix. Additionally, the application performance and dough lipid profiling of the ‘No-lyso-phospholipase’ CRC08319 was tested in comparison.

The Crusty Roll baking was done according to ‘Crusty Roll’ description presented in the ‘Assay and Methods’ section above.

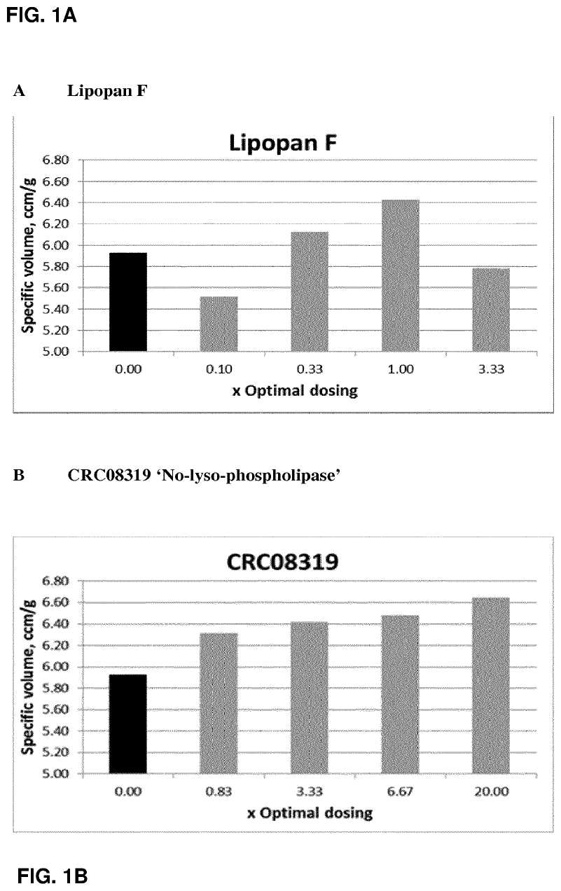

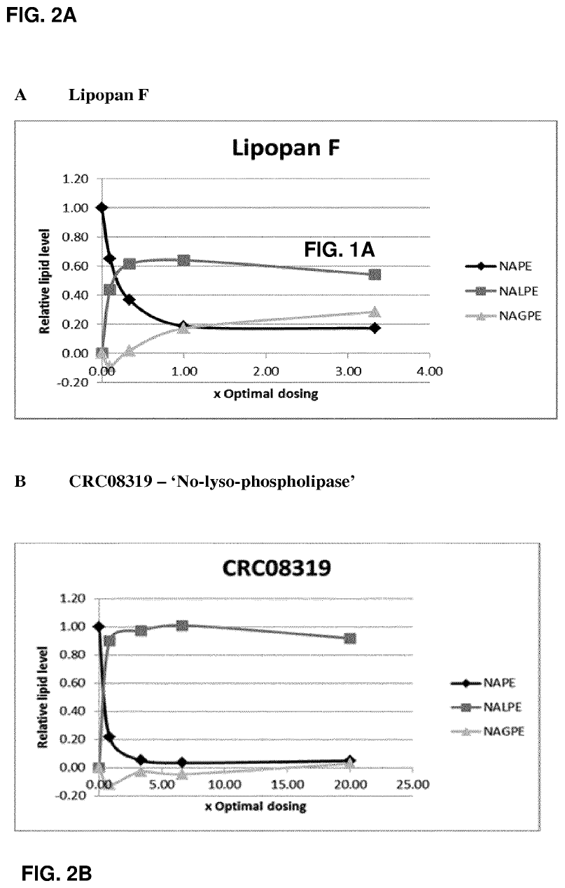

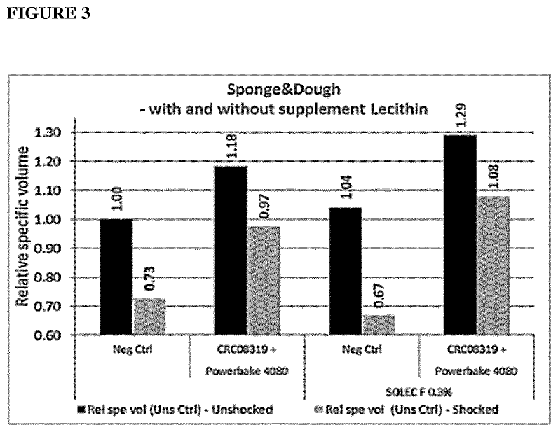

The experimental setup of the application trials and the results from the baking evaluation as well as dough lipid profiling is presented in respectively Table 5 (A and B), FIGS. 1(A and B) and FIGS. 2A and B).

### Table 5A and B. Experimental Setup of Enzyme Dose-Response Test in Crusty Roll Baking

All dosages are presented as dosage relative to the optimal dosage of Lipopan F (relative based on mg protein/kg flour). The optimal dosage of Lipopan F is defined as the dosage giving the highest specific volume in the presented baking setup. The optimal Lipopan F dosage is presented by ‘1’, Negative controls is presented by ‘0’.

For example, Lipopan F dose-response Trial 2 (Table 5A): A Lipopan F dosage of 0.10 reflects that Lipopan F dosage in this trial was ‘0.10×Optimal dosage of Lipopan F’—or in other words, that Lipopan F dosage in this trial was 10% of the dosages used in the trial showing the optimal dosage of Lipopan F (the trial showing the highest specific volume (Trial 4)).

TABLE 5A
Crusty roll bake-Lipopan F dose-response

| Trial | Trial | Trial | | Trial |
|---|---|---|---|---|
| 1 | 2 | 3 | Trial 4 | 5 |

Dosage relative to optimal dosage of Lipopan F

| | | | | | |
|---|---|---|---|---|---|
| Lipopan F dose-response | 0 | 0.10 | 0.33 | 1.00 | 3.33 |
| (× Optimal dosing | | | | (optimal Lipopan | |
| (Lipopan F)) | | | | F dosage) | |

TABLE 5B
Crusty roll bake-CRC08319 dose-response

| Trial | Trial | Trial | Trial | Trial |
|---|---|---|---|---|
| 1 | 2 | 3 | 4 | 5 |

Dosage relative to optimal dosage of Lipopan F

| | | | | | |
|---|---|---|---|---|---|
| CRC08319 dose-response | 0 | 0.83 | 3.33 | 6.67 | 20 |
| (× Optimal dosing | | | | | |
| (Lipopan F)) | | | | | |

### FIG. 1. Crusty Roll Specific Volume (ccm/g) Presented as Function of Optimal Dosage of Lipopan F

Optimal dosage of Lipopan F is defined as Lipopan F dosage giving the highest specific volume in the presented baking setup—and optimal Lipopan F dosage is presented by ‘1’. All other dosages presented are relative to the optimal Lipopan dosage (based on mg protein/kg flour). 0 represents Negative control.

Lipopan F show optimal dosage represented by ‘1×Optimal dosing’. With increasing dosage Lipopan F show overdosing presented by a decrease in specific volume. In contrast, increasing dosage of CRC08319 show continued increase or leveling out in specific volume.

Fully fermented doughs were frozen, freeze dried and lipids in the dry dough were extracted with water saturated butanol and analyzed by HPLC according to procedure described in Assays and Methods. Results are show in FIG. 2.

### FIG. 2. Dough Lipid Profiling

Application effects on specific volume are supported by lipid profile. Current marketed product—Lipopan F—show hydrolysis of NAPE to NALPE, and at higher dosages further hydrolysis of NALPE to NAGPE aligning to a decrease in specific volumes.

With 80% hydrolysis of NAPE (NAPE reduced to 20% of start level (Start level=0×Optimal dosing (Negative ctrl)) Lipopan F show NALPE generation of around 60%. This 80% hydrolysis of NAPE and 60% generation of NALPE correlates with optimal dosage (highest specific volume=1×Optimal dosage) of Lipopan F. Lipopan F shows alignment between specific volume and peak in NALPE levels. For Lipopan F it is evident that the peak in NALPE levels around 60% is followed by reduction in NALPE at the higher dosages tested (dosages above optimal dosage (1)) aligning with formation of NAGPE. The highest levels of NAGPE are observed at the highest dosage Lipopan F.

In contrast, the ‘No-lyso-phospholipase’—CRC08319—show full conversion of NAPE to NALPE. At 80% hydrolysis of NAPE (NAPE reduced to 20% of start level), NALPE levels are at 80%. With further hydrolysis of NAPE the ‘No-lyso-phospholipase’ show a continued increase or leveling out in NALPE levels which is also aligned with specific volume.

With full hydrolysis of NAPE (>90-95% hydrolyzed) reaction equilibrium starts to show with continued increase or leveling out of the NALPE levels.

Even when the ‘No-lyso-phospholipase’ is dosed 20×optimal dosage of Lipopan F corresponding to 4-6 fold the dosage of ‘No-lyso-phospholipase’ resulting in complete NAPE hydrolysis (˜10% residual NAPE) NAGPE levels are still below 5%.

### Example 20. Application of No-Lyso-Phospholipases to Lipid Containing Food Matrix

No-lyso phospholipase can for example be used in egg yolk and whole eggs, in processed meats, in degumming of vegetable oils, in milk products like cheese, and in bakery products such as bread and in bakery products such as sweet bakery goods, including cakes and cookies.

### Egg Yolk Containing Products

Egg yolk is well known for use in the food industry due to its emulsifying properties. Approximately 30% of the lipid in egg yolk is phospholipid, which contributes to egg yolks emulsification properties. In many foods including mayonnaise, sauces, dressings and cakes the emulsifying properties of egg yolk are exploited. For some food applications, however, the emulsification properties of egg yolk are not sufficient to obtain a homogenous product without separation. In mayonnaise, for example, pasteurization of the product at high temperatures cause the product to separate. No-lyso phospholipase may be used to modify phospholipid to lyso-phospholipid in egg yolk (and food products containing egg yolk). Product separation at high temperature pasteurization can be avoided using enzyme modified egg yolk.

### Processed Meat Products

No-lyso phospholipase may be used in processed meat products. No-lyso phospholipase will contribute to improve the emulsification of processed meat products and contribute to better consistency and reduced cooking loss. No-lyso phospholipase added to processed meat will convert meat phospholipids to lysophospholipids. Because of the emulsifying properties of lysophospholipids, this component contributes to improved consistency and less cooking induced loss by improved emulsification of the fat in the meat.

### Vegetable Oil

Crude vegetable oils like soya bean oil contain 1-2% phospholipids. Phospholipids are removed from the oil during the refining process, to improve the quality of the oil and prevent sedimentation in the oil. The removal of phospholipids is conducted by a so-called degumming process during the oil reefing process. The degumming can be conducted by chemical or enzymatic means. In the degumming process ‘No-lyso phospholipase’ may be used to convert phospholipids to lysophospholipids which are more water-soluble and can be removed from the oil by washing with water. Enzymatic hydrolysis of phospholipids is a gentler process compared with the chemical degumming which requires harsh alkaline or acidic conditions. Degumming with No-lyso phospholipase will cause fewer effluents.

### Milk Products

No-lyso phospholipase may be used in milk products. No-lyso phospholipase will contribute to increased yield during cheese production. No-lyso phospholipase added to milk will convert milk phospholipids to lysophospholipids. Because of the emulsification properties of lysophospholipids, this will contribute to increased cheese yield by entrapping more lipid in the cheese curd.

### Sweet Bakery Goods

Eggs are a substantial part of most cake products. No-lyso phospholipase may be used to modify the phospholipids in egg by production of lyso-phospholipids, which contribute to improved emulsification during cake mixing and gives a softer and more tender crumb. No-lyso phospholipase may also be used directly in the cake dough to modify the phospholipids of the flour.

1-101. (canceled)

102. A method of modification of a phospholipid emulsifier comprising treatment of the emulsifier with an enzyme comprising an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02, and/or a NALPE/NAPE activity ratio of less than 0.12.

103. The method of claim 102 wherein the sn1/sn2 specificity ratio is about 60/40, 70/30, 80/20, 90/10, 95/5 or 99/1.

104. The method of claim 103 wherein said phospholipid emulsifier is lecithin or lysolecithin.

105. The method of claim 102 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

106. A method of creating a lysophospholipid in a lipid containing food matrix comprising adding to the lipid containing food matrix an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02 and/or a NALPE/NAPE activity ratio of less than 0.12.

107. The method of claim 106 wherein the sn1/sn2 specificity ratio is about 60/40, 70/30, 80/20, 90/10, 95/5 or 99/1.

108. The method of claim 106 wherein said lipid containing food matrix is selected from the group consisting of eggs and food products containing eggs, dough for sweet bakery goods, processed meat, milk based products, and vegetable oil.

109. The method of claim 108 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

110. A dough comprising an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02, and/or a NALPE/NAPE activity ratio of less than 0.12.

111. The dough of claim 110 wherein the sn1/sn2 specificity ratio is about 60/40, 70/30, 80/20, 90/10, 95/5 or 99/1.

112. The dough of claim 111 having improved dough extensibility and/or stability.

113. The dough of claim 112 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

114. The dough of claim 113 further comprising at least one additional enzyme useful for improving dough and/or a baked product made therefrom.

115. The dough of claim 114 wherein the additional enzyme is selected from the group consisting of amylase, cyclodextrin glucanotransferase, peptidase, transglutaminase, lipase, galactolipase, phospholipase which is different than said phospholipase A1, cellulase,
hemicellulase, protease, protein disulfide isomerase, glycosyltransferase, peroxidase, lipoxygenase, laccase, and oxidase.

116. A method of preparing a baked product comprising baking a dough according to claim 110.

117. A baked product obtainable by the method according to claim 116.

118. The baked product of claim 117 having at least one improved property selected from the group consisting of improved crumb pore size, improved uniformity of gas bubbles, no separation between crust and crumb, increased volume, increased crust crispiness and improved oven spring.

119. The baked product according to claim 118 wherein the improved property is crust crispiness.

120. A pre-mix for baking comprising flour and an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02 and/or a NALPE/NAPE activity ratio of less than 0.12.

121. The pre-mix of claim 120 wherein the sn1/sn2 specificity ratio is about 60/40, 70/30, 80/20, 90/10, 95/5 or 99/1.

122. The pre-mix of claim 121 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

123. The pre-mix of claim 122 further comprising at least one additional enzyme useful for improving dough and/or a baked product made therefrom.

124. The pre-mix of claim 123 wherein the additional enzyme is selected from the group consisting of amylase, cyclodextrin glucanotransferase, peptidase, transglutaminase, lipase, galactolipase, phospholipase which is different than said phospholipase A1, cellulase, hemicellulase, protease, protein disulfide isomerase, glycosyltransferase, peroxidase, lipoxygenase, laccase, and oxidase.

125. A baking improver comprising a granulate or agglomerated powder comprising an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02 and/or a NALPE/NAPE activity ratio of less than 0.12.

126. The baking improver of claim 125 wherein the sn1/sn2 specificity ratio is about 60/40, 70/30, 80/20, 90/10, 95/5 or 99/1.

127. The baking improver of claim 126 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

128. The baking improver of claim 127 further comprising at least one additional enzyme useful for improving dough and/or a baked product made therefrom.

129. The baking improver of claim 128 wherein the additional enzyme is selected from the group consisting of amylase, cyclodextrin glucanotransferase, peptidase, transglutaminase, lipase, galactolipase, phospholipase which is different than said phospholipase A1, cellulase, hemicellulase, protease, protein disulfide isomerase, glycosyltransferase, peroxidase, lipoxygenase, laccase, and oxidase.

130. An isolated polypeptide comprising a phospholipase A1 characterized by having an sn1/sn2 specificity ratio of about 55/45 or greater wherein said phospholipase A1 has a lysophospholipase/phospholipase activity ratio of less than 0.02 and/or a NALPE/NAPE activity ratio of less than 0.12.

131. The isolated polypeptide of claim 130 wherein the sn1/sn2 specificity ratio is about 60/40, 70/30, 80/20, 90/10, 95/5 or 99/1.

132. The isolated polypeptide of claim 131 wherein said phospholipase A1 is an enzyme comprising a protein sequence having at least 80% sequence identity to SEQ ID NO: 2, SEQ ID NO: 4, SEQ ID NO: 6, SEQ ID NO: 8, SEQ ID NO: 10 SEQ ID NO: 12, SEQ ID NO: 14 or SEQ ID NO: 16.

133. An isolated polynucleotide comprising a nucleic acid sequence encoding the isolated polypeptide of claim 132.

134. A recombinant expression vector comprising a polynucleotide according to claim 133.

135. A host cell comprising the recombinant expression vector of claim 134.

136. A method of making a dough, said method comprising admixing a dough component selected from the group consisting of flour, salt, water, sugar, fat, lecithin, oil emulsifier and yeast with an isolated polypeptide comprising a phospholipase A1 characterized by having an sn1/sn2 specificity ratio of about 55/45 or greater wherein said phospholipase A1 has a lysophospholipase/phospholipase activity ratio of less than 0.02, and/or a NALPE/NAPE activity ratio of less than 0.12.


SEQUENCE LISTING (26 sequences)

SEQ ID NO: 1 - protein, 186 aa, Trichoderma harzianum
MKFAALLATLAPAVLALPASDAALTRRQTSLSTITDQYLFSLTLPDFISRRNAKNPATLDWTSDGCTSSPDNPFGFPFVPACYRHDFGYQNYRIQNRFTESGKLSIDNNFKADLYFQCQTSSVQSVCNALADVYYAAVRAFGGGDASPGKREQSQEDLVKVYEEKLEIYNNAVKDAQDKGLLPILE

SEQ ID NO: 2 - protein, 170 aa, Trichoderma harzianum
LPASDAALTRRQTSLSTITDQYLFSLTLPDFISRRNAKNPATLDWTSDGCTSSPDNPFGFPFVPACYRHDFGYQNYRIQNRFTESGKLSIDNNFKADLYFQCQTSSVQSVCNALADVYYAAVRAFGGGDASPGKREQSQEDLVKVYEEKLEIYNNAVKDAQDKGLLPILE

SEQ ID NO: 3 - protein, 180 aa, Pestalotiopsis fici
MRFISTLSLCILPAAALAVPVADKRQDDVEAVTDEILFDITLPEFTTRRNAEDPSYLDWTSDGCTDSPDNPLGFPYEPACNRHDFGYTNYREQSRFTVSAKASIDSNFKDDLYYQCEVNGSFESICEALADVYYAAVVEFGGDDATPGKRSSLYEEKLAIYNQLVAEAVAKGELVLPETA

SEQ ID NO: 4 - protein, 162 aa, Pestalotiopsis fici
VPVADKRQDDVEAVTDEILFDITLPEFTTRRNAEDPSYLDWTSDGCTDSPDNPLGFPYEPACNRHDFGYTNYREQSRFTVSAKASIDSNFKDDLYYQCEVNGSFESICEALADVYYAAVVEFGGDDATPGKRSSLYEEKLAIYNQLVAEAVAKGELVLPETA

SEQ ID NO: 5 - protein, 177 aa, Metarhizium guizhouense
MKLNTTLLALATTALAAPASDVTTPKRQDINTVTDQLLFSSTLSQFEARRNAKEPPSLDWSSDGCTSSPDNPFGFPFLPACHRHDFGYQNYRIQKRFTKAAKAKIDSNFKTDLYYQCRSVSAKDACNGLADVYYEAVKEFGGGDATKRDRSDYDRAVAAYNAAVKEAQEQGLLPILD

SEQ ID NO: 6 - protein, 161 aa, Metarhizium guizhouense
APASDVTTPKRQDINTVTDQLLFSSTLSQFEARRNAKEPPSLDWSSDGCTSSPDNPFGFPFLPACHRHDFGYQNYRIQKRFTKAAKAKIDSNFKTDLYYQCRSVSAKDACNGLADVYYEAVKEFGGGDATKRDRSDYDRAVAAYNAAVKEAQEQGLLPILD

SEQ ID NO: 7 - protein, 184 aa, Diaporthe ampelina
MKVSSIACVAWLVPATFALGPVPLSPRQDIQTVTDSYLFDISLAQFITYRDAQNPSTLDWTSDGCSDSPDNPLGFNFEPACYRHDFGYTNYRAQSRFTKAAKASIDTNFQEDLKFQCESESFESICDALADVYYTAVKLFGGQDATKRADSEDVDADALAEYEHAVAVYEQLVAEAKANGEIPA

SEQ ID NO: 8 - protein, 166 aa, Diaporthe ampelina
LGPVPLSPRQDIQTVTDSYLFDISLAQFITYRDAQNPSTLDWTSDGCSDSPDNPLGFNFEPACYRHDFGYTNYRAQSRFTKAAKASIDTNFQEDLKFQCESESFESICDALADVYYTAVKLFGGQDATKRADSEDVDADALAEYEHAVAVYEQLVAEAKANGEIPA

SEQ ID NO: 9 - protein, 187 aa, Magnaporthe oryzae
MAGDNVGFGLMKFLALLAAAVTVLASPLQMERRQSDLVAITDKLLYSTSLPDFVARRNARDPPSLDWTSDGCTSSPDNPLGFPFTPACNRHDFGYQNYRIQSRFTQSNKFNIDNNFLLDLNNQCNGLNIIARGTCRALADVYYAAVRAFGGSDATPGKRNEDLEKEYNEKLAIYNVLLAEAKKGNYI

SEQ ID NO: 10 - protein, 162 aa, Magnaporthe oryzae
SPLQMERRQSDLVAITDKLLYSTSLPDFVARRNARDPPSLDWTSDGCTSSPDNPLGFPFTPACNRHDFGYQNYRIQSRFTQSNKFNIDNNFLLDLNNQCNGLNIIARGTCRALADVYYAAVRAFGGSDATPGKRNEDLEKEYNEKLAIYNVLLAEAKKGNYI

SEQ ID NO: 11 - protein, 184 aa, Neonectria ditissima
MKFTAPFLVFLSGAAAFPAREIASSVVGRATIQETTDELLFSVTLPQFTVRRNALNPPTLDWTSDGCTSSPDNPFGFPFVPACNRHDFGYNNYRIQTRFTVSAKAKIDSNFKTDLYYQCSSVSATSVCNALADVYYAAVRAFGGDDATPGKRTEDLVKIYEEKVAIYNSLVEEAQKNGELPILE

SEQ ID NO: 12 - protein, 168 aa, Neonectria ditissima
FPAREIASSVVGRATIQETTDELLFSVTLPQFTVRRNALNPPTLDWTSDGCTSSPDNPFGFPFVPACNRHDFGYNNYRIQTRFTVSAKAKIDSNFKTDLYYQCSSVSATSVCNALADVYYAAVRAFGGDDATPGKRTEDLVKIYEEKVAIYNSLVEEAQKNGELPILE

SEQ ID NO: 13 - protein, 187 aa, Trichoderma gamsii
MKLATLFFTLAPAALALPASKESPTRRQSDLVAITDQLLFNTTLPDFITHRNAQDPSTLDWTSDGCTDSPDNPFGFPYVPACNRHDFGYQNYRLQNRFTDSGKLNIDNNFKSDLYYQCQSVSAQSACEDLADVYYAAVRAFGGGDSSPGRRDESHEDLVKEYEAKLEIYHQAVKEAQEKGLLPILDQ

SEQ ID NO: 14 - protein, 171 aa, Trichoderma gamsii
LPASKESPTRRQSDLVAITDQLLFNTTLPDFITHRNAQDPSTLDWTSDGCTDSPDNPFGFPYVPACNRHDFGYQNYRLQNRFTDSGKLNIDNNFKSDLYYQCQSVSAQSACEDLADVYYAAVRAFGGGDSSPGRRDESHEDLVKEYEAKLEIYHQAVKEAQEKGLLPILDQ

SEQ ID NO: 15 - protein, 178 aa, Metarhizium anisopliae
MKFNNAILLALATTVLAAPASDVTTPKRQDINTVTDQLLFSSSLPQFEARRNANDPPLLDWSSDGCTSSPDNPFGFPFLPACHRHDFGYQNYRIQTRFTKAAKAKIDSNFKSDLYYQCQSVSAKNACDRLADVYYEAVKEFGGGDATKRDRSDFDRAVAAYNAAVKEAQEQGLLPVLD

SEQ ID NO: 16 - protein, 161 aa, Metarhizium anisopliae
APASDVTTPKRQDINTVTDQLLFSSSLPQFEARRNANDPPLLDWSSDGCTSSPDNPFGFPFLPACHRHDFGYQNYRIQTRFTKAAKAKIDSNFKSDLYYQCQSVSAKNACDRLADVYYEAVKEFGGGDATKRDRSDFDRAVAAYNAAVKEAQEQGLLPVLD

SEQ ID NO: 17 - protein, 279 aa, Fusarium heterosporum
EAEAAVGVTSTDFTNFKFYIQHGAAAYCNSGTAAGAKITCSNNGCPTIESNGVTVVASFTGSKTGIGGYVSTDSSRKEIVVAIRGSSNIRNWLTNLDFDQSDCSLVSGCGVHSGFQNAWAEISAQASAAVAKARKANPSFKVVATGHSLGGAVATLSAANLRAAGTPVDIYTYGAPRVGNAALSAFISNQAGGEFRVTHDKDPVPRLPPLIFGYRHTTPEYWLSGGGGDKVDYAISDVKVCEGAANLMCNGGTLGLDIDAHLHYFQATDACNAGGFSWR

SEQ ID NO: 18 - protein, 346 aa, Fusarium oxysporum
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 19 - DNA, 558 bp, Trichoderma harzianum
  source 1..558: mol_type = other DNA
atgaagttcgccgccctgctggccaccctggcccccgccgtcctggccctccccgcctctgacgccgccctcacccgacgacagacctctctgagcaccatcaccgaccagtacctgttcagcctcaccctccctgacttcatctctcgacgaaacgccaagaaccctgccaccctggactggacctctgacggctgcaccagctctcctgacaaccctttcggcttccctttcgtccccgcctgctaccgacacgacttcggctaccagaactaccgcatccagaaccgattcaccgagtccggcaagctcagcatcgacaacaacttcaaggccgacctgtacttccagtgccagaccagctccgtccagtctgtctgcaacgccctggccgacgtctactacgccgccgtccgagccttcggcggcggcgacgccagccccggcaagcgcgagcagtctcaggaggacctcgtcaaggtctacgaggagaagctggagatctacaacaacgccgtcaaggacgcccaggacaagggcctgctgcctatcctcgag

SEQ ID NO: 20 - DNA, 540 bp, Pestalotiopsis fici
  source 1..540: mol_type = other DNA
atgcgcttcatcagcaccctgtctctgtgcatcctccccgccgccgccctcgccgtccccgtcgccgacaagcgccaggacgacgtcgaggccgtcaccgacgagatcctgttcgacatcaccctgcctgagttcaccacccgacgcaacgccgaggacccttcttacctggactggacctctgacggctgcaccgactctcctgacaaccctctgggcttcccttacgagcctgcctgcaaccgacacgacttcggctacaccaactaccgcgagcagtctcgattcaccgtctctgccaaggccagcatcgactctaacttcaaggacgacctgtactaccagtgcgaggtcaacggctctttcgagtctatctgcgaggccctcgccgacgtctactacgccgccgtcgtcgagttcggcggcgacgacgccacccccggcaagcgatctagcctgtacgaagaaaagctggccatctacaaccagctggtcgccgaggccgtcgccaagggcgagctggtcctccctgagaccgcc

SEQ ID NO: 21 - DNA, 531 bp, Metarhizium guizhouense
  source 1..531: mol_type = other DNA
atgaagctcaacaccaccctcctggccctggccaccaccgccctcgccgcccctgcctctgacgtcaccacccctaagcgacaggacatcaacaccgtcaccgaccagctgctgttcagctctaccctgtctcagttcgaggcccgacgaaacgccaaagagcctccttctctggactggtcttctgacggctgcacctctagccctgacaaccctttcggcttccctttcctccccgcctgccaccgacacgacttcggctaccagaactaccgcatccagaagcgattcaccaaggccgccaaggccaagatcgactctaacttcaagaccgacctgtactaccagtgccgcagcgtctctgccaaggacgcctgcaacggcctcgccgacgtctactacgaggccgtcaaggaatttggcggcggcgacgccaccaagcgagaccgatctgactacgaccgagccgtcgccgcctacaacgccgccgtcaaagaggcccaggaacagggcctgctccccatcctggac

SEQ ID NO: 22 - DNA, 552 bp, Diaporthe ampelina
  source 1..552: mol_type = other DNA
atgaaggtctcctctattgcctgcgttgcttggctcgtccccgctaccttcgccctgggccccgtccctctgtctcctcgccaggacattcagaccgttaccgactcttacctgttcgacattagcctggctcagttcatcacctaccgagacgcccagaaccctagcaccctggactggacctccgacggctgcagcgacagccccgacaaccctctgggcttcaacttcgagcctgcttgctaccgccacgacttcggctacaccaactaccgcgctcagtctcgattcaccaaggccgctaaggcttctattgacaccaacttccaggaagacctcaagttccagtgcgagtctgagtctttcgagtccatttgcgacgctctcgccgacgtctactacaccgccgttaagctgttcggcggccaggacgccaccaaacgagctgactccgaggacgttgacgctgacgctctcgctgagtacgagcacgctgttgccgtttacgagcagctcgttgctgaggccaaggctaacggcgagatccctgcc

SEQ ID NO: 23 - DNA, 561 bp, Magnaporthe oryzae
  source 1..561: mol_type = other DNA
atggcgggtgacaacgtcggcttcggcctgatgaagttcctcgctctcctcgccgctgctgttaccgttctggcttctcctctgcagatggagcgccgacagtctgacctcgtcgccattaccgacaagctgctgtactctaccagcctccccgacttcgttgctcgccgaaacgctcgtgaccctcctagcctggactggaccagcgacggctgcacctctagccctgacaaccctctgggcttccctttcacccccgcttgcaaccgccacgacttcggctaccagaactaccgcatccagtctcgattcacccagtctaacaagttcaacattgacaacaacttcctgctcgacctgaacaaccagtgcaacggcctgaacatcattgcccgaggcacctgccgcgctctggctgacgtttactacgctgccgtccgcgctttcggcggcagcgacgccacccccggcaagcgaaacgaggacctggagaaagagtacaacgagaagctcgctatatataacgttctcctggctgaggccaagaagggcaactacatc

SEQ ID NO: 24 - DNA, 552 bp, Neonectria ditissima
  source 1..552: mol_type = other DNA
atgaagttcactgctcctttcctggttttcctcagcggcgccgctgccttccccgctcgcgagattgcatctagcgtcgttggccgcgctaccatccaagagaccactgacgagctcctgttctctgttactctgcctcagttcaccgtccgccgaaacgctcttaaccctcccactcttgactggacttctgacggctgcactagcagccctgacaaccctttcggcttccctttcgtccccgcttgcaaccgacacgacttcggctacaacaactaccgcatccagactcgattcaccgttagcgctaaggccaagatcgactctaacttcaagaccgacctgtactaccagtgctctagcgtctctgccacctccgtttgcaacgccctggctgacgtttactacgctgctgtccgagctttcggcggcgacgacgccacccccggcaagcgaactgaggacctggttaagatctacgaagagaaggtcgccatctacaacagccttgttgaggaagcccagaagaacggcgagcttcctattcttgag

SEQ ID NO: 25 - DNA, 561 bp, Trichoderma gamsii
  source 1..561: mol_type = other DNA
atgaagctggctaccctgttcttcaccctcgcccctgctgctctggctctccccgcttctaaagaatctcctacccgccgacagtctgacctcgttgctatcaccgaccagctcctgttcaacaccaccctgcctgacttcattacccaccgaaacgctcaggaccctagcaccctcgactggacctctgacggctgcaccgactctcctgacaaccctttcggcttcccttacgtccccgcttgcaaccgccacgacttcggctaccagaactaccgactgcagaaccgattcaccgacagcggcaagctgaacattgacaacaacttcaagagcgacctgtactaccagtgccagtccgttagcgcccagagcgcttgcgaggacctcgctgacgtttactacgccgctgttcgcgctttcggcggcggcgacagcagccccggccgccgagacgagtctcacgaggacctcgttaaggaatacgaggccaagctggagatctaccaccaggccgtcaaagaagctcaagaaaagggcctcctgcctattctggaccag

SEQ ID NO: 26 - DNA, 534 bp, Metarhizium anisopliae
  source 1..534: mol_type = other DNA
atgaagttcaacaacgccatcctcctggctctcgccaccaccgttctcgccgcccccgcttctgacgtcaccacccctaagcgacaggacattaacaccgttaccgaccagctcctgttctctagcagcctccctcagttcgaggctcgccgaaacgctaacgaccctcctctgctggactggtctagcgacggctgcacctctagccctgacaaccctttcggcttccctttcctgcctgcttgccaccgacacgacttcggctaccagaactaccgcattcagacccgattcaccaaggccgctaaggctaagattgacagcaacttcaagtctgacctgtactaccagtgccagagcgtttccgccaagaacgcttgcgaccgactcgctgacgtttactacgaggccgtcaaagaatttggcggcggcgacgctaccaagcgagaccgatctgacttcgaccgcgctgttgctgcttacaacgctgccgttaaggaagcccaagagcagggcctgctccccgtcctggac